(19) United States
(12) Patent Application Publication
(10) Pub. No.: US 2004/0005571 A1
(43) Pub. Date: Jan. 8, 2004

(54) Axs gene and protein and methods related thereto

(76) Inventors: R. Scott Hawley, Overland Park, KS (US); Joseph J. Kramer, Davis, CA (US)

Correspondence Address: POLSINELLI SHALTON & WELTE, P.C., 700 W. 47TH STREET, SUITE 1000, KANSAS CITY, MO, 64112-1802, US

(21) Appl. No.: 10/189,718

(22) Filed: Jul. 3, 2002

(51) Int. Cl. A01N 61/00; C12Q 1/68; C07H 21/04; C12N 9/22; C12P 21/02; C12N 5/06; C07K 16/40

(52) U.S. Cl. 435/6; 435/69.1; 435/320.1; 435/348; 435/199; 530/358; 530/388.26; 536/23.2; 514/1

(57) ABSTRACT

The present invention relates to methods for using an Axs gene and related mutants, as well as the proteins or amino acid sequences expressed therefrom. The present invention includes methods for predicting meiotic nondisjunction using an Axs mutant, as well as methods for destroying defective oocytes, using either an Axs mutant or an Axs gene. The present invention pertains to methods for selecting wild-type gametes in individuals with chromosomally based pathologies through the use of the Axs, and Axs related nucleic acid sequences and the proteins therefrom.

### FIELD OF INVENTION

The present invention is related to an isolated Axs gene and mutant Axs genes, as well as proteins or amino acid sequences expressed therefrom. In particular, the present invention relates to methods for isolating and using the Axs gene and related mutant alleles, as well as the amino acid sequences or molecules related thereto, and expressed therefrom.

### BACKGROUND OF INVENTION

Meiosis is the process of doubling the genetic chromosome number, with it known that meiosis involves a single chromosomal duplication followed by two successive nuclear divisions. The end result of meiosis is the formation of a gamete, which is a haploid germ cell. More specifically, the gamete is a sex cell, either spermatozoan or egg (ovum). Each gamete will receive one member of each chromosome pair. The ability to study and understand meiosis is important to a number of research areas. Knowledge of the mechanisms of meiosis can be used in identifying birth defects (thereby possibly preventing such defects), practicing birth control, or promoting fertilization. Thus, it is desired to have an improved understanding of meiosis. It is further desired to have methods, which can be used to disrupt meiosis as a method of birth control, as well as methods for use in predicting events which may lead to birth defects or sterility.

Drosophila melanogaster, as well as other species of Drosophila, are commonly known as the “fruit fly.” This species is a well-known model organism used in the study of genetics. In particular, this species is well suited as a model organism for the study of specific genes in multicellular development and behavior. The fruit fly's haploid genome contains about 165 million nucleotide pairs. Of these nucleotide pairs, about 110 million base pairs are unique sequences present in the euchromatin. The fruit fly is thought to contain about 15,000 genes, about 1,000 of which have been cloned, with the transcripts ranging in size from 0.3-15 thousand base pairs. Many of these genes are homologous (of a similar sequence) to genes found in mammals and other organisms. As such, it is known that phenotypic and genotypic information developed from the study of Drosophila can be used to predict phenotypic and genotypic characteristics in, for example, mammalian cells and organisms. Generalized knowledge of Drosophila genetics can be applied to the overall study of genetics and, in particular, the study of meiosis.

Drosophila melanogaster includes, as part of its genome, a gene known as Abnormal X segregation (Axs). Axs expresses a spindle associated transmembrane protein. The gene and the protein expressed therefrom (the amino acid sequence) have been isolated and sequenced. Some of the resultant phenotypic characteristics associated with the Axs gene have been observed and recorded in numerous scientific articles.

The following is an explanation of meiosis in Drosophila and the role of Axs. In many, if not most animals, oocyte meiosis proceeds without the benefit of centrioles (a cellular organelle which organizes microtubules). Microtubules are long, nonbranching, thin cylinders with an outside diameter of about 24 nanometers (nm) and a central lumen about 15 nm in diameter. The tubules are composed of strands called protofilaments, and there are usually 13 of these. Each protofilament in turn is composed of a linear array of subunits, and each subunit is a dimer containing an alpha and a beta tubulin molecule. Microtubules play key roles in cell division, secretion, intracellular transport, morphogenesis, and ciliary and flagellar motion. Meiosis without centrioles is known as anastral meiosis and includes the formation of anastral spindles. In order to understand the mechanism by which anastral spindles function, it is desired to elucidate the processes inherent in the establishment of bipolarity, the construction and maintenance of spindle poles, and mechanisms of spindle elongation. It is especially desired to understand the genes and proteins, which effect these processes.

The chromosomes of Drosophila oocytes are organized in a specialized chromatin structure known as the karyosome. The chromatin is the complex of nucleic acids (DNA and RNA) and proteins (histones and nonhistones) comprising eukaryotic chromosomes. The chromatin is packaged within a specialized nucleus termed the germinal vesicle. The chromatin exists at this stage in a postsynaptic state which corresponds to prophase of the meiotic cell cycle. Upon entry into meiotic metaphase I the germinal vesicle breaks down and meiotic spindle assembly begins. Meiotic spindle formation begins with the establishment of an array of microtubules lacking a defined pole that emanate from the major chromosomes. As prometaphase continues, these bundles of microtubules are sculpted together on each side of the metaphase plate (the metaphase plate is the grouping of the chromosomes in a plane at the equator of the spindle during the metaphase stage of meiosis (or mitosis)) to form a bipolar spindle, which is a collection of microtubules responsible for the movement of eukaryotic chromosomes during mitosis or mitosis. Meiosis will then proceed, whereby the chromosome pairs will separate and move toward each pole.

To date, three proteins have been identified in Drosophila oocytes that function in the formation of spindle poles: a microtubule motor Ned (Mattheis et al) and two highly conserved microtuble associated proteins (MAPs): d-TACC and Msps (Cullen and Ohkura, 2001). As pointed out by Theurkauf (2001), genetic and cytological studies support a model in which the Ned motor transports Msps to the developing poles of the spindle. Msps then recruits d-TACC to form a novel structure that stabilizes the spindle poles.

Observations that Ncd⁺ oocytes can build bipolar spindles, albeit unstable ones, imply that additional functions are also required to create the bipolar spindle. Thus, other genes involved in this process likely remain to be identified. Mutants in the genes encoding all three of these proteins produce multi-polar spindles. This phenotype has also been observed in mutations of the Axs locus, but only in the presence of more than one pair of achiasmate chromosomes.

Phenotypic studies have revealed that the protein expressed from the Axs gene helps to facilitate proper chromosome disjunction (moving apart of chromosomes) during female meiosis. In particular, the Axs protein contributes to meiotic spindle formation, which is necessary for disjunction to occur. If the spindle is not formed properly, nondisjunction results. Nondisjunction is the failure of homologous chromosomes (in meiosis I, primary nondisjunction) or sister chromatids (in meiosis II) to separate properly and move to opposite poles. Nondisjunction results in one daughter cell receiving both and the other daughter cell none of the chromosomes in question. While it has been observed that a mutated Axs causes nondisjunction, the fact that Axs is critical to spindle assembly was not previously known. As a result, it is desired to have a composition, probe, or method for analyzing spindle formation, as it relates to Axs. It is further desired to have an agent, whether it be gene, protein, or small molecule, which can inhibit or promote spindle formation.

There are at least four known mutant alleles of the Axs gene, including the Axs^{D} allele. All of the mutations are generally referred to as Axs^{D}. The mutations affect the female meiotic chromosome and are semi-dominant meiotic mutants. The dominant allele affects the distributive pairing, whereby disruption of meiotic segregation of non-exchange chromosomes is affected. Axs^{D} does not affect the frequency of exchange. Disruption of the segregation of achiasmate homologs is affected by Axs^{D} (achiasmate relates to the absence of crossing over of homologous chromosomes).

It was previously determined that although Axs^{D} had little or no effect on the frequency or distribution of exchange, or on the disjunction of exchange bivalents, nonexchange X-chromosomes undergo nondisjunction at high frequencies in Axs^{D}/+ and Axs^{D}/Axs^{D} females. The symbol “+” relates to wild-type. This increased Xchromosome nondisjunction was shown to be a consequence of an Axs-induced defect in distributive segregation.

Further, studies have shown that in Axs^{D}-bearing females, fourth chromosome nondisjunction was observed only in the presence of nonexchange X-chromosomes and was thought to be the result of improper X and fourth chromosome associations within the distributive system. In XX females bearing a compound fourth chromosome, the frequency of non-homologous disjunction of the X-chromosomes from the compound fourth chromosome was shown to account for at least 80% of the total X nondisjunction observed. In addition, Axs^{D} diminished or ablated the capacity of nonexchange X-chromosomes to form trivalents in females bearing either a Y chromosome or a small free duplication for the X. Axs^{D} also impaired compound X from Y segregation. The effect of Axs^{D} on these segregations paralleled the defects observed for homologous nonexchange X-chromosome disjunction in Axs^{D} females. In addition to its dramatic effects on the X-chromosome, Axs^{D} was observed to exert a similar effect on the segregation of a major autosome and the obligate achiasmate fourth chromosome.

Thus, Axs^{D} has been identified as a female-specific meiotic mutation exhibiting high levels of nondisjunction of nonrecombinant chromosomes at meiosis I. Both dominant and recessive mutations at the Axs locus caused achiasmate homologs to nondisjoin at high frequency, but the segregation of chiasmate bivalents is not affected. Although nondisjoining Xs are free to undergo heterologous disjunctions in Axs oocytes, the heterologous associations do not cause nondisjunction. This conclusion is based on the observation that the frequency of Axs^{D}-induced X-chromosome nondisjunction (˜35%) is independent of the number or identity of the heterologous achiasmate chromosomes that are present. Rather, the number and type of available heterologs appear to influence only the fraction of X-chromosome nondisjunction that is due to heterologous segregations.

From the above, it is known that Axs is critical to spindle formation and, resultingly, disjunction. It is desired to have methods, compositions, and kits for identifying the allelic state of the Axs gene in a host organism. It is further desired to have methods, compositions, and kits for identifying the expressed Axs proteins and mutant Axs proteins, as all of this information can be used to predict nondisjunction, which relates to sterility and birth defects. The isolated gene and protein can also be used in the study of related genes and proteins in similar or related organisms, in particular, organisms having a common ancestor. This information would also be useful towards the design of agents which cause or reverse sterility and/or birth control.

Both birth defects and sterility can be the results of the spindle not forming and nondisjunction occurring. Errors in chromosome segregation result in about 45% of the cases where a host has an Axs^{D} gene, and sterility occurs in about 90% of these cases. As would be expected, the allelic state of Axs is a predictor of chromosomal nondisjunction and sterility. Thus, it is desired to have, for example, a cDNA probe for detecting Axs^{D} and similar alleles. It is also desired to have an antibody probe for detecting the Axs^{D} amino acid sequence or protein, as well as amino acid sequences of related alleles. Because of the above problems, it is desired to have a system and method for examining nondisjunction and its causes. It is desired to have the capability to use the wild-type Axs gene and protein as well as the corresponding mutant genes and proteins. It is further desired to understand how the transmembrane protein expressed by Axs functions, and why the mutant allelic forms of those proteins prevent proper bipolar spindle assembly.

### SUMMARY OF INVENTION

The present invention relates to the Axs gene and related alleles, as well as the various amino acid molecules or sequences expressed therefrom. In particular, the present invention relates to at least one isolated nucleic acid molecule, and the expressed amino acid molecule, which inhibits or prevents proper female meiotic spindle assembly, in particular, bipolar spindle assembly. Additionally, the present invention relates to various compositions and methods, which utilize the Axs nucleic acid molecule and related nucleic acid molecules, as well as mutant alleles of Axs and the corresponding alleles of genes related to Axs. The amino acid sequences expressed therefrom are also a part of this invention.

The various nucleic acid molecules and amino acid sequences can be used as part of a birth control method to produce defective gametes, or as part of a method for predicting birth defects. More particularly, the molecules and sequences can be used to promote or inhibit nondisjunction and to predict the nondisjunction event. Additionally, these agents can be used as part of a method, which serves to increase the likelihood of normal progeny from reproductively compromised individuals including those individuals which exhibit a variety of chromosomally based diseases affecting meiosis. Trisomic individuals exhibiting any one of several known or possible viable, non-sterile trisomies occurring in human populations are candidates for this approach. In addition, individuals heterozygous for chromosomal translocations are also likely to benefit from such methodologies. These women produce disomic ova at high frequencies as a result of improper chromosome alignments at metaphase. By introducing Axs^{D} product into the ova of such women, prior to ovulation, it might be possible to select against such aneuploidy-generating ova.

The available isolated nucleic acid molecules include SEQ. ID NOs. 1, 2, and 3, and complementary sequences thereof. Also, degenerate variants of the listed sequences may be used. Isolated nucleic acid molecules that encode a protein or amino acid sequence similar to and having the same function as that expressed by Axs^{D} or a related mutant allele, according to the previously listed sequences, and variants thereof may also be used. Related to the isolated nucleic acid molecule and useful herewith are sequences, which are at least 50% homologous to the nucleic acid molecules. Alternatively, isolated nucleic acid molecules can be used that are at least 60%, 75%, or 90% homologous to the above nucleic acid molecules.

Expression vectors, which prevent or hinder female meiotic spindle assembly, can be formed from a promoter operably linked to one of the above listed nucleic acid molecules. The vector can be used as part of a method for producing a protein or amino acid sequence that prevents or inhibits female meiotic spindle assembly. The method includes culturing a cell, which contains the vector, under conditions and for a time sufficient to produce the protein or amino acid sequence. Thus, for example, a viral vector capable of directing expression of one of the above nucleic acid molecules can be used.

The present invention also relates to a transfected host germ cell carrying a vector formed according to the discussed method and formed from one of the nucleic acid molecules previously discussed. More particularly, the transfected host cells will be oocytes. Host organisms, which include a transfected oocyte, are also part of the present invention. Any host organism can be transfected, as long as it produces gametes via a meiotic process. Isolated oligonucleotides that bind to any of the above nucleic acid molecules may be used herewith. The oligonucleotides can be used as part of a kit or method for identifying Axs mutant alleles.

As would be expected, not only are the isolated nucleic acid molecules important, but so are the isolated proteins or amino acid sequences, which, for example, prevent or inhibit female meiotic spindle assembly. The available proteins include isolated proteins, such as SEQ. ID NOs. 4, 5, and 6, and proteins encoded by the previously discussed nucleic acid molecules.

Proteins or amino acid sequences that are either 50%, 60%, 75%, or 90% homologous with the listed amino acid sequences are also part of the present invention. More importantly, the proteins should function the same as the above proteins. Related thereto, and included herewith, are antibodies, which specifically bind to the proteins or amino acid sequences. The available antibodies include an antibody that binds specifically to a protein expressed from wild-type Axs, Axs^{D}, or one of the other mentioned alleles, an antibody that selectively binds to an epitope in the amino-terminal extramembrane domain of the wild-type and Axs^{D} protein. It is also desired to possess an antibody, which alters the function of the wild-type Axs protein such that it functions analogously to that of the Axs^{D} protein. Towards this end, we are generating antibodies to the loop and transmembrane region adjacent to the position of the Axs^{D} mutation. Hybridomas that express the antibodies are further related to the present invention.

A probe for identifying a protein that causes or promotes meiotic failure is desired. Such a probe is derived from one of the discussed proteins. A cDNA probe can be formed from an isolated nucleic acid taken from one of the previously mentioned nucleotide sequences. The cDNA should be at least 50% homologous, and more preferably 90% homologous, to one of the disclosed nucleic acid molecules. Alternatively, an RNA probe can be formed.

Besides the mutant alleles, mutant nucleic acid molecules, and the expressed mutant amino acid sequences, the non-mutant Axs nucleic acid molecules and the expressed amino acid sequences may also be used herewith. In particular, nucleic acid molecule SEQ. ID NO. 7, and amino acid sequence, SEQ. ID NO. 8, may be used. Probes, vectors, transfected cells, transfected host organisms, oligonucleotides, and hybridomas, which use or incorporate the Axs nucleic acid molecule or related nucleic acid molecules, and amino acid sequences expressed therefrom are also part of the present invention.

The various nucleic acid molecules and amino acid sequences can be used as part of a variety of different methods. One such method relates to preventing or inhibiting female meiotic spindle assembly. The method is practiced by expressing an Axs^{D} gene or other mutant allele to form an Axs^{D} protein or amino acid sequence and supplying the protein to a germ cell or oocyte during the first meiotic division, in order to prohibit or inhibit female meiotic spindle assembly. The oocyte can be derived from an insect, such as Drosophila, or a non-human animal. Mammals, including humans, could also be treated with this method. All organisms, which possess nucleic acid sequences related to the Axs gene are candidates for such a methodology. Expression can be controlled by injecting an Axs^{D} protein encoding nucleic acid molecule, or the Axs^{D} protein itself, into the oocyte. The expression can also be controlled by delivery of an Axs^{D} nucleic acid or protein molecule by micro-vessels. Conversely, a small molecule, which binds to an endogenous Axs protein to create a defect parallel to that generated by the Axs^{D} mutant, can be used to the same end.

Another method relates to predicting spindle formation during female meiosis I. This method includes determining the allelic state of the Axs gene or related sequences in the organism in question. Such a determination is practiced using one of several methodologies. These include using the wild-type or mutant Axs cDNA or the cDNAs of related genes as probes in hybridization procedures designed to detect single or multiple base pair mutations present in the sample of interest.

Alternatively, mutations can be identified through a combination of PCR amplification and direct sequencing of the Axs or Axs related gene derived from a test individual's genomic DNA. This procedure is preferred as it represents an unbiased approach towards identifying both known and novel alleles of Axs or genes related to Axs and offers the ability to identify mutations in regulatory regions (e.g., splice sites, promoters). Depending on their nature, identification of mutations within Axs or Axs related genes may indicate a high likelihood that proper spindle formation will not occur during female meiosis I, and nondisjunction will result. For example, identification of the Axs^{D} mutation in the Axs gene or the corresponding mutation in an Axs related gene would suggest a high rate of nondisjunction in the test female. Again, the female can be of any of a variety of origins, including of mammalian origin.

Another method relates to affecting meiotic spindle assembly in order to increase the rate of normal progeny production in individuals that harbor chromosomal abhorations. Affected individuals include those who are trisomic for individual chromosomes (e.g., Down's syndrome patients), or those that are heterozygous for chromosomal translocations or inversions. Individuals of this class of genotypes exhibit meiosis I nondisjunction at frequencies greatly exceeding those found in genotypically normal individuals. The rate at which these individuals exhibit nondisjunction is unique to, and dependent upon the detailed structure of the abhoration in question. Gametes derived from these meioses, however, are aneuploid or polyploid and can result in early embryonic lethality of their offspring, or in the case of trisomic females, reconstitution of the parental genotype with respect to the chromosome in question. Further, the affected chromosomes of this genotypic class are likely segregated according to the human equivalent of the distributive pairing system of which Axs is an essential component. This genotypic class is decidedly sensitive to perturbation of the distributive system, and in particular, perturbation of Axs function represented by Axs^{D} and other Axs allelic forms.

Application of Axs^{D}, or agents which subvert wild-type Axs to function in a manner similar to that of Axs^{D}, to oocytes derived from individuals of this genotypic class would result in the inhibition of proper meiotic spindle assembly in those oocytes. Such oocytes would withdraw from the meiotic cell cycle and undergo atresia or apoptosis. As mentioned, oocytes, which have chromosomal aberrations, are more sensitive to Axs^{D} than individuals who are “normal.” In diploid organisms which undergo meiosis, the production of the chromosomal substrate of meiosis I is dependent upon a series of mitoses occurring in the germline. These divisions are somewhat error-prone and they themselves are known to be responsible for the generation of pathologies associated with defects in chromosomal number and structure. Individuals of the genotypic class described above, undergo these same divisions and exhibit segregation defects at a rate at least equal to, but likely greater than that observed in individuals with a normal genetic complement. One of the daughter products from these defective mitoses is what is considered to be a “normal” or wild-type genetic complement. For example, through these mitotic errors an individual trisomic for a particular chromosome would yield a normal disomic oocyte. Similarly, inversion heterozygotes will yield both oocytes homozygous for the inversion or homozygous wild-type chromosome. In both of these cases, the chromosomes are segregated by the normal chiasmate based system, and importantly, they would not be sensitive to Axs^{D} or agents which phenocopy the effects of Axs^{D}. Thus, application of such agents would serve as a strong selective agent leading to the preeminence of gametes with a normal chromosomal complement in individuals of this genotypic class.

Methods for predicting nondisjunction during female meiosis I can be practiced. This includes forming an Axs^{D} or Axs mutant allele antibody probe specific to mutant forms, and contacting the antibody probe with an oocyte. Attachment of the probe indicates the presence of a mutant gene, thereby indicating the likelihood that nondisjunction will occur.

A method of purifying an Axs^{D} or Axs mutant protein, or amino acid sequence from a biological sample containing Axs^{D} or related protein can be practiced. This involves providing an affinity matrix comprising one of the above discussed antibodies bound to a solid support, contacting the biological sample with the affinity matrix to produce an affinity matrix Axs^{D} protein complex, and separating the affinity matrix Axs^{D} protein complex from the remainder of the biological sample. The Axs^{D}, or mutant protein is then released from the affinity matrix. The above methods can also be practiced with the non-mutant Axs.

Kits can be developed from the present invention. A kit for detecting an Axs^{D} gene or similar mutant allele can be formed from a container and a nucleic acid molecule comprising the nucleotide molecules previously discussed. Also, a kit for detecting an Axs^{D} protein or similar mutant amino acid sequence can be made from a container and the protein of interest.

Meiotic spindle formation is initiated during meiosis. To prevent spindle formation and disjunction, a protein can be attached to a meiotic sheath protein to inhibit or prevent disjunction. The protein is an amino acid sequence molecule related to the above Axs^{D} and mutant allele sequences. As such, the present invention relates to amino acid sequences, which bind to meiotic sheath proteins and result in nondisjunction.

In summary, it has been discovered that mutations in the Axs gene behave as dosage sensitive antimorphs that disrupt the process of achiasmate segregation in Drosophila oocytes, as well as homologs thereof. Molecular analysis of the Axs gene indicates that it encodes the founding member of a new gene family of predicted transmembrane proteins. Germline expression of the mutant Axs^{D} or similar allele under the control of an inducible promoter results in the ablation of the bipolar spindle assembly and the random segregation of achiasmate chromosomes. No such effect is observed upon germline expression of the wild-type Axs^{{+}} protein. Immuno-localization studies during oogenesis position the Axs protein on or near the nuclear envelope of nurse cells as it is deposited in the growing oocyte. Moreover, in both oocytes and synticial embryos, the Axs protein appears to be organized by microtubules. In late stage prophase oocytes, the Axs protein localizes to the prophase oocyte nuclear membrane. However, upon GV breakdown, the Axs protein is localized to a membranous or vesicular sheath that surrounds the meiosis I spindle midzone. The ability of the mutant Axs protein to disrupt spindle assembly and chromosome segregation demonstrates that the sheath-like structure and its associated proteins play an important role in meiotic spindle assembly and function.

The present invention is advantageous because it can be used to predict a nondisjunction event, which is associated with sterility and birth defects. As such, the present invention can be used as a method to predict the possibility of sterility or the occurrence of a birth defect. The present invention can also be used to promote sterility as part of a birth control method. Moreover, the present invention can be used as a selective agent for normal progeny in individuals harboring chromosomally based genetic disease. Finally, the present invention is an advantageous research tool for studying meiosis.

### BRIEF DESCRIPTION OF DRAWINGS

The application file contains at least one drawing executed in color. Copies of this patent application publication with color drawing(s) will be provided by the Office upon request and payment of the necessary fee.

FIG. 1a is a recombination map showing the location of Axs, CG13010, CG13011, and CG9699 genes proximal to rudimentary(r);

FIG. 1b illustrates the position of an Axs gene relative to constructs SCX1, SCR2, SCX2, and XS1;

FIG. 2a is a printout illustrating various transmembrane segments as determined by a hydrophibicity analysis, associated with Axs using a Tmpred program;

FIG. 2b shows the molecular lesions for Axs mutants, in view of the eight transmembrane segments found in Axs;

FIG. 3 is a photomicrograph showing the failure of spindle microtubules to organize into bundled arrays, both tubulin and histone were used;

FIGS. 4a-4d are photomicrographs of the Axs protein which show the spindle forming, the DAPI photo is of the chromosomes, the tubulin shows the spindle, Axs shows the Axs localizing to the spindle;

FIG. 5 is a photomicrograph of GFP-Axs fusion, localizing to a perinuclear region;

FIG. 6 is a photomicrograph of a wild-type Axs protein associated with spindle microtubules, t stands for time in seconds, live imaging of Axs and tubulin was used to show the cell cycle, also Axs localization to the spindle is shown;

FIG. 7 is a schematic showing the position of the known Axs mutation;

FIG. 8 shows data comparing various mutants defective for achiasmate segregation, nondisjunction of chromosomes 4 and X are demonstrated going to opposite poles;

FIG. 9 shows data comparing expression of Axs with over-expression of Axs^{D} and the impact on nondisjunction;

FIG. 10 is a photomicrograph of stage 14 meiotic spindles in Axs^{D/X} oocytes, a defect in assembly is shown;

FIG. 11a shows a wild type meiotic spindle and a Axs mutant allele spindle stained with DAPI, tubilin, TACC, and the merge of the three stains;

FIG. 11b is a graph which shows the spindle length of the wild-type and mutant with different stains again used;

FIG. 12 is a photomicrograph of a wild-type spindle disjunction event stained with anti-tubulin and anti-histone;

FIG. 13 is the same as FIG. 12 except the disjunction event is shown from start to finish; and,

FIG. 14 is a model of the plasmid UAS showing the insert site for Axs cDNA.

### DETAILED DESCRIPTION

The present invention relates to at least one nucleic acid molecule, and at least one protein or amino acid sequence, which prevent or inhibit female meiotic spindle assembly and, resultingly, cause or promote nondisjunction to occur during meiosis. The present invention also relates to nucleic acid molecules and amino acid sequences, which can be used to detect or predict the likelihood of correct or incorrect female meiotic spindle assembly. Specifically, the present invention relates to and includes isolated mutant Axs nucleic acid molecules and related genes, as well as the non-mutant Axs gene or nucleic acid molecule, and amino acid sequences expressed therefrom. The Axs and mutant proteins are transmembrane proteins.

Further, the present invention relates to expression vectors, which are formed from the mentioned nucleic acid molecules, and host germ cells, which have been transfected with the vectors. The present invention relates to isolated oligonucleotides that bind to one of the nucleic acid molecules. Antibodies which specifically bind to the proteins, and probes for isolating the proteins or nucleic acid molecules are further part of the present invention.

Yet another part of the present invention relates to methods for preventing or inhibiting female meiotic spindle assembly, methods for predicting spindle formation, and methods for predicting nondisjunction during female meiosis I. The present invention relates to methods for purifying the Axs^{D} and similar mutant allele proteins and kits for detecting the Axs^{D} gene and other Axs mutant alleles, as well as the related proteins or amino acid sequences. Such kits can also be used with non-mutant nucleic acid molecules and amino acid sequences. As such, the present invention can be used to both predict and promote or inhibit nondisjunction during female meiosis I. Finally, the present invention relates to the use of Axs mutants as selective agents for normal progeny in individuals harboring chromosomally-based genetic disease.

As stated, the Axs gene naturally occurs in insects, specifically Drosophila, and mammals, including humans. It is hypothesized that the mutant alleles occur in these same species. The Drosophila Axs gene, or nucleic acid molecule, is identified as SEQ. ID NO. 7, and the protein expressed therefrom is identified as SEQ. ID NO. 8. In the presence of a non-mutant Axs gene, disjunction will occur normally during meiosis, assuming that no other genetic or external causes impact the meiotic process. Axs expresses an Axs transmembrane protein, which causes bipolar spindle assembly to occur and which, in turn, contributes to the process of disjunction.

As used here, an amino acid sequence is at least two amino acids attached to each other. Use of the term amino acid sequence will include peptides, polypeptides, and proteins. The nucleic acid molecule will be formed from at least two nucleotides attached to one another. The nucleic acid molecule can be either DNA or RNA, as well as any other related nucleic acid molecules. The nucleic acid molecule will include oligonucleotides, genes, and groups of genes. It is preferred to use the Axs or Axs^{D} gene.

Mutant alleles of the Axs gene express a protein or amino acid sequence that will prevent or inhibit female meiotic spindle assembly during meiosis and, as such, will cause nondisjunction. Host cells that harbor chromosomal aberrations are sensitive to the Axs mutants. As discussed, expression of such a mutant can be desirable, especially when used as a method of birth control. Birth control is achieved because gametes that are aneuploidy are produced. The isolated nucleic acid molecules, which prevent or inhibit female meiotic spindle assembly are referred to as the Axs mutant alleles. These nucleic acid molecules include Axs^{D}, Axs^{r1}, and Axs^{r2}. Additionally, there is an Axs^{r3} molecule; however, the nucleotide sequence and expressed amino acid sequence are not listed herein. Axs^{D} is the most effective nucleic acid molecule at preventing female meiotic spindle assembly and, as such, is the preferred mutant allele for use in the present method and invention. The isolated nucleic acid molecules for each of the mutant alleles are listed as SEQ. ID NOs. 1-3. Throughout the application, Axs^{D} or mutant alleles will be referenced. The Axs^{D} or mutant alleles will include Axs^{D}, Axs^{r1}, Axs^{r2}, and Axs^{r3}, and any related mutant alleles.

Complementary sequences to the previously listed nucleic acid molecules may also be used with the present invention. The complementary sequences may be for the mutant or non-mutant Axs gene or nucleic acid molecule. Complementary sequences are base sequences of polynucleotides related by base pairing rules. As would be expected, a complementary sequence is one that can be expressed to form a protein or amino acid sequence that prevents nondisjunction, or causes nondisjunction to result, dependent upon the desired outcome. A complementary sequence to the non-mutant will cause disjunction. A complementary sequence to the mutant will prevent or inhibit disjunction. Further, degenerate variants of the sequences may be used. Degenerate variants are those that code for the same amino acid sequence. Essentially, any isolated nucleic acid molecules that encode an Axs mutant protein or amino acid sequence may be used in the present invention. Nucleic acid molecules that express a transmembrane protein which causes nondisjunction may be used.

Nucleic acid molecules homologous to mutant allele nucleic acid molecules, and the non-mutant nucleic acid molecules, may be used to prevent or cause meiotic spindle formation or assembly. The selected homologous sequence is again dependent upon the desired outcome. Homologous nucleic acid molecules are identified by using the Psi blast (REF: Altschul, S. F., Madden, T. L., Schäffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402) to generate alignments. Suitable homology will include those nucleic acid molecules that are 50% homologous to the listed mutant alleles, or non-mutants. More preferably, the homology will be 60% and, even more preferably, 75% homologous to the mutant alleles, or non-mutants. The most preferred homologous nucleic acid molecule will be 90% homologous to the mutant alleles, or non-mutants, in particular, Axs^{D} and Axs. Homologous nucleic acid molecules may be derived from mammals, including humans, as well as other geneses, such as insects. Homology refers to nucleic acid molecules or amino acid sequences which are similar because they have a number of nucleotides or amino acids which are the same or share similar biochemical properties.

Isolated oligonucleotides can be derived from the nucleic acid molecules. The oligonucleotides will be the active portions of the nucleic acid molecules that prevent spindle assembly. Such oligonucleotides can be used with the present invention. The active region, which forms the oligonucleotide molecules, includes those oligonucleotide molecules identified by a screen spanning the length of the Axs^{D} gene as being able to cause significant nondisjunction. Conversely, oligonucleotides related to the non-mutant gene can be used to cause disjunction.

Expression vectors, which prevent or inhibit female meiotic spindle assembly, can be formed from the above-discussed nucleic acid molecules, using known procedures. The preferred procedures include using various inducible promoters, or the Axs promoter itself. A promoter will be operably linked to the isolated nucleic acid molecule to form the expression vector. Any promoter, which causes expression of the nucleic acid molecule and inducible promoters can be used. A UAS vector, for example, can be used. It is further preferred to include a marker with the vector, such as an ampicillin marker. Suitable vectors include shuttle vectors which permit growth of vector DNA in bacterial cells. Once grown and prepared this DNA can be used for introduction into the host of interest. Specific preferred vectors include pUASP for use in insect cells or virally based vectors for mammalian systems.

Once the vectors are formed, they can be used to transfect a host cell, whereby a transgenic host germ cell will be produced that incorporates a vector that expresses the selected nucleic acid molecule, which prevents or inhibits female meiotic spindle assembly. The method for transfecting the host germ cell is well known, and comprises culturing the vectors with the host germ cells. The preferred methods for insects include using P-element mediated transformation. The host cell can be of any of a variety of origins, including mammalian- or insect-derived cells. More preferably, the host cells are derived from non-human mammals and humans. Once the nucleic acid molecules are expressed in the host cell, the resultant proteins can prevent the spindle assembly. If a non-mutant is used, spindle assembly occurs.

A transgenic animal can be formed using the present invention. In particular, transgenic non-human animals can be formed by inserting the nucleic acid molecules into a host germ cell and allowing that germ cell to undergo fertilization and undergo normal development. The conditions and requirements for normal development are well studied and are dependent upon the host organism in question.

The proteins or amino acid sequences expressed by the mutant alleles, the non-mutants, and the listed nucleic acid molecules can prevent or inhibit spindle assembly and can be isolated and purified and used in methods to promote nondisjunction. Additionally, the non-mutant proteins or amino acid sequences can be isolated and used to promote normal disjunction. The proteins or amino acid sequences from the non-mutant nucleic acid molecules can also be isolated and purified. Such isolation and purification include the use of known procedures and methods, including affinity chromatography or purification. The isolated proteins include those listed herein as SEQ. ID NOs. 4, 5, and 6. Additional, suitable proteins or amino acid sequences include those encoded by a mutant allele Axs nucleic acid molecule (such as Axs^{D}), and proteins, which are 90% homologous with the proteins of SEQ. ID NOs. 4, 5, and 6. Proteins that are 50% homologous to the proteins of SEQ. ID NOs. 4, 5, and 6 may also be used, with proteins 60% homologous more preferred. A protein that is 75% homologous to SEQ. ID NOs. 4, 5, and 6 is even more preferred. As such, any of a variety of proteins may be used, as long as they are expressed by an Axs mutant allele or homologous nucleic acid molecule or degenerate variant, and prevent female meiotic spindle assembly. Resultingly, the proteins will cause or promote nondisjunction. The Axs protein of Drosophila is SEQ. ID NO. 8. Non-mutant, homologous amino acid sequences may be used to promote and cause disjunction. The homology will be the same as mentioned.

Probes, which can be used to isolate the above proteins and/or genes, can be formed from such proteins or genes. The probes include cDNA, mRNA, and monoclonal and polyclonal antibodies. All the probes are formed using known procedures. Probes, which are 50% homologous to the proteins or amino acid sequence, may be formed. More preferably, the probes will be 75% and, even more preferably, 90% homologous to the above proteins or amino acid sequences. The method used to determine homology uses alignments of similar sequences derived from the Psi-Blast program(Altschul, S. F., Madden, T. L., Schäffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402.).

Antibodies, which specifically bind to the above-listed proteins, are part of the present invention. Additionally, hybridomas that produce such antibodies are used herewith. In addition to protein probes, cDNA probes may be formed, which are comprised of isolated nucleic acid molecules previously discussed. As such, any antibody, which binds specifically to an Axs mutant allele or non-mutant, may be used. Antibodies that selectively bind to an epitope in the putative ligand-binding domain of the Axs mutant protein may be used. A non-mutant epitope may also be used.

The isolated proteins or amino acid sequences may be used as part of a method for preventing or inhibiting female meiotic spindle assembly. The proteins or amino acid molecules can be used as part of a method for inhibiting or preventing spindle formation in species where anastral meiosis occurs. Once the mutant allele is expressed to form the protein or amino acid sequence, such protein can be isolated and purified. The isolated and purified protein, or amino acid sequence, is then contacted with an oocyte or germ cell during prophase I, whereby female meiotic spindle assembly is prohibited. The oocyte may be derived from any of a variety of species, including insects and mammals. More preferably, the oocyte is derived from a Drosophila, human, livestock, dog, or cat species. As such, non-human animals may be treated with the isolated protein. The protein is placed in contact with the oocyte by way of any of a variety of different methods. As such, any method is suitable that allows the protein to contact the oocyte and, thereby prevent or inhibit female meiotic spindle assembly.

As stated, germ cells from a variety of species may be transfected using a vector. Other methods can be used to transfect a host germ cell and express the desired gene. For example, a calcium phosphate system may be used. An injection process may be used, whereby a nucleic acid molecule is injected into the oocyte, and the molecule is then expressed to produce the desired protein. Expression of the mutant protein will prevent or inhibit spindle assembly in the germ cell. Germ cells containing achiasmate chromosomes are expected to be particularly sensitive to such treatment and as a result will undergo apoptosis or atresia. Microinjection processes are known and can be practiced with the present technology. An alternative method involves delivery via a micro-vessel, which is also known in the field.

The proteins and genes can be used as part of a method related to affecting meiotic spindle assembly in order to increase the rate of normal progeny production in individuals that harbor chromosomal abhorations. Individuals who can be treated include those who are identified as trisomic for individual chromosomes (e.g., Down's syndrome patients), or those that are heterozygous for chromosomal translocations or inversions. Individuals of this class of genotypes exhibit meiosis I nondisjunction at frequencies greatly exceeding those found in genotypically normal individuals. The rate at which these individuals exhibit nondisjunction is unique to, and dependent upon the detailed structure of the abhoration in question. Gametes derived from these meioses are aneuploid or polyploid and can result in early embryonic lethality of their offspring, or in the case of trisomic females, reconstitution of the parental genotype with respect to the chromosome in question. Further, the affected chromosomes of this genotypic class are likely segregated according to the human equivalent of the distributive pairing system of which Axs is an essential component. This genotypic class is decidedly sensitive to perturbation of the distributive system, and in particular, perturbation of Axs function represented by Axs^{D} and other Axs allelic forms.

Application of Axs^{D}, or agents which subvert wild-type Axs to function in a manner similar to that of Axs^{D}, to oocytes derived from individuals of this genotypic class would result in the inhibition of proper meiotic spindle assembly in those oocytes. Such oocytes would withdraw from the meiotic cell cycle and undergo atresia or apoptosis. Individuals of the genotypic class exhibit segregation defects at a rate at least equal to, but likely greater than that observed in individuals with a normal genetic complement. One of the daughter products from these defective mitoses is what is considered to be a “normal” or wild-type genetic complement. The method includes identifying an individual predisposed to this genotype. An Axs^{D} protein can be contacted with an oocyte in prophase I. This will promote nondisjunction and select for mitotic errors and against trisomic oocytes. Thus, application of such agents would serve as a strong selective agent leading to the preeminence of gametes with a normal chromosomal complement in individuals of this genotypic class.

A method of utilizing a vector having a mutant allele nucleic acid molecule may also be used to prevent or inhibit spindle assembly. The vector will transfect the oocyte. A small molecule that binds to an endogenous Axs mutant protein to create a defect parallel to that generated by an Axs^{D} mutant may also be used.

The isolated nucleic acid molecules, in particular the Axs mutant alleles, may be used to form gene probes that can predict spindle formation during female meiosis I. In particular, the Axs gene probe may be contacted with DNA from a homolog, whereby an attachment of the probe indicates the presence of a mutant gene analog. This will indicate a high likelihood that spindle formation will not occur during female meiosis I, and nondisjunction will result. Alternatively, PCR amplifcation followed by direct sequencing can be used to determine the allelic state of the Axs or Axs related gene in question in test individuals. Primers for such a determination will depend upon the gene and organism in question, however, they may include those designed to detect mutational lesions in both the coding and non-coding region of those genes. These tests may be used with both insects and mammals.

A method for predicting spindle formation during female meiosis I has been developed from the present invention. This is accomplished by isolating an Axs gene and forming an Axs gene probe. The gene probe is then contacted with DNA from a homolog, whereby an attachment of the probe indicates the presence of a normal gene analog in the homolog of the Axs gene. This indicates a high probability that spindle formation will occur during female meiosis I and disjunction will result.

A similar method for predicting nondisjunction can be practiced. Here the method includes forming an Axs^{D} or mutant allele antibody probe specific to mutant form and contacting the antibody probe with an oocyte. Attachment of the probe indicates the presence of a mutant gene, thereby indicating nondisjunction will occur.

As would be expected, methods for purifying the Axs^{D} protein or similar mutant protein from a biological sample containing Axs^{D} protein, or mutant protein, are important. A preferred method includes providing an affinity matrix with the antibody to Axs^{D} bound to a solid support. A biological sample is contacted with the affinity matrix, to produce an affinity matrix-Axs^{D} protein complex. Next, the affinity matrix-Axs^{D} protein complex is separated from the remainder of the biological sample, with the Axs^{D} protein released from the affinity matrix.

A kit for detecting an Axs^{D} gene or related nucleic acid molecule can be formed. The kit will preferably have a container and a nucleic acid molecule, which includes any of SEQ. ID NOs. 1-3. The kit is a hybridization kit.

A kit for detecting the allelic state of an Axs or Axs related gene can be formed. Such a kit will consist of a container and a set of PCR primers spanning the Axs or Axs related gene in question. The kit will preferably include the necessary components to conduct PCR. The kit will preferably include options for sequencing the PCR products. In addition, such a kit will contain a positive control consisting of a nucleic acid molecule representing a wild-type allele of the gene in question. For example, SEQ. ID NO. 7, the Axs cDNA or a genomic clone spanning the Axs gene may be used as the control.

A kit for detecting an Axs^{D} protein or amino acid molecule can also be formed. The kit will preferably have a container and a purified antibody preparation specifically recognizing the Axs^{D} or the corresponding mutant form of an Axs related protein in question. Proteins known to react with the antibody will be included as a positive control for such procedures. For example, polypeptides derived from SEQ. ID NOs. 4-6 would serve such a purpose.

A method for destroying defective oocytes can be practiced. The method includes forming an Axs^{D} antibody probe specific to mutant forms and contacting the antibody probe with an oocyte. Attachment of the probe indicates the presence of a mutant gene, thereby indicating nondisjunction will occur. The oocyte is then destroyed by any conventional means.

A kit for detecting an Axs gene or related nucleic acid molecule can be formed. The kit will preferably have a container and a nucleic acid molecule, which includes SEQ. ID NO. 7. The kit is a hybridization kit.

A kit for detecting an Axs protein or amino acid molecule can also be formed. The kit will preferably have a container and a purified antibody preparation recognizing the wild-type, or normal, form of the Axs or Axs related protein. Positive control protein will also be included in such a kit. For example, polypeptides derived from SEQ. ID NO. 8 would serve such a purpose.

A method can be practiced, whereby trisomic individuals, or individuals heterozygous for chromosomal translocations or inversions are identified and treated. In the method, the individuals who are likely to have this genotype are first identified. Such individuals will be female. Once identified, oocytes are harvested and isolated from the female for treatment in vitro. After isolation, the germ cells can be treated with an Axs^{D} protein or related mutant protein or amino acid sequence. This will select against the trisomic individuals. In particular, mitotic errors in the oocyte are selected for, which will result in a population that is normal or has a wild-type genetic complement. The normal oocytes can then be fertilized in vitro. This method can be used as a model in insects, non-human mammals, and, potentially, humans.

The present invention relates to a meiotic spindle structure formed from a meiotic sheath protein and an Axs protein that localizes to the sheath protein, whereby disjunction is promoted. The sheath protein/Axs protein complex will promote chromosome segregation.

Finally, the present invention relates to a family of proteins used to promote disjunction. The proteins are characterized by eight transmembrane domains. The protein will localize to the meiotic sheath.

### EXAMPLES

### Example 1

Standard molecular techniques were used to isolate and construct gene transfer constructs disclosed herein. (Sambrook et al., 1989). The source of genomic DNA 30 kb proximal to r was a genomic clone provided by Nicholas Brown, University of Cambridge, Cambridge, United Kingdom.

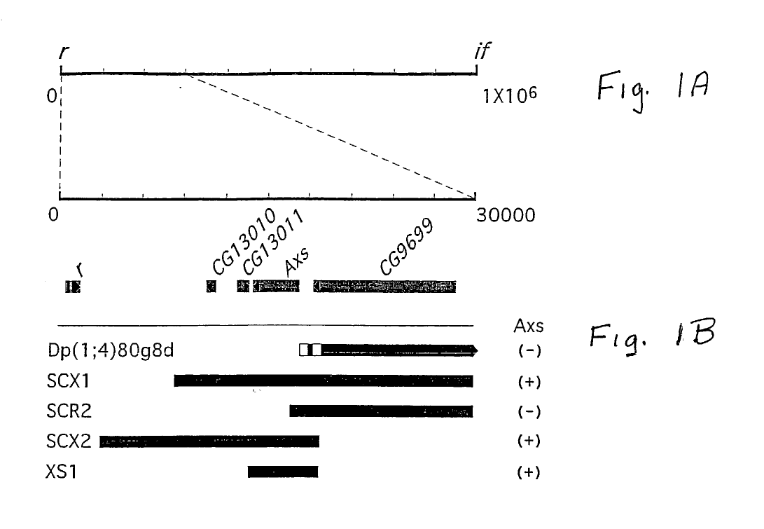

This is roughly illustrated in FIG. 1a. Segments of this region were subcloned into pCASPER4 and introduced into the germline via P-element mediated transformation (Rubin, GM and Spradling, AC—Vectors for P element-mediated gene transfer in Drosophila. Nucleic Acids Res Sep. 24, 1983;11(18):6341-51).

Axs cDNA's were derived from an existing ovarian cDNA library previously constructed by the inventor but not published. Information regarding the Axs^{D} and its intragenic suppressors was obtained by directly amplifying PCR products from male genomic DNA harboring the appropriate chromosome. Designation of the mutation for each allele as well as its respective parent chromosome was based upon direct sequencing of three independent PCR reactions.

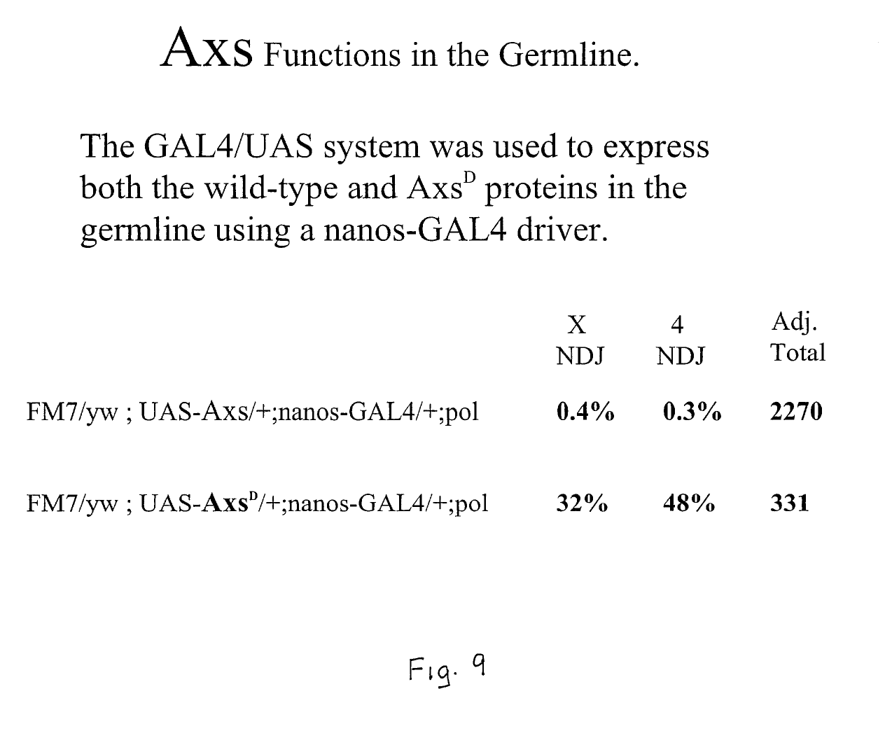

For the UAS constructs, a cDNA incorporating the Axs^{D} mutation was constructed by PCR, and both Axs cDNA and its Axs^{D} derivative were subcloned into the germline UAS vector, pUASp (Rorth, 1997). This is shown at FIG. 14. Axs^{MYC} and Axs^{GFP} fusions were constructed as amino terminal fusions of three copies of the myc epitop (CEQKLISEEDL) recognized by hybridoma 9E10 (Covance, Inc.) and eGFP (Clontech, Inc.), respectively. UAS designates upstream activator sequence and corresponds to enhancer binding sites recognized by the GAL4 activator. UAS sequences are contained within a vector that can be used to transform Drosophila melanogaster. The results are illustrated in FIG. 9.

### Example 2

Standard fly husbandry was used for the construction and maintenance of stocks used herein (Ashburner, 1989). Genetic tests for meiotic nondisjunction have been described previously (Hawley et al., 1993). GAL4 drivers used in this study were obtained from the Bloomington stock center and are as follows:

w¹¹¹⁸; P {w^{+mC}=GAL4::VP16-nanos.UTR}MVD1

y¹w*; P {w+mC=Act5C-GAL4} 17bFO1/TM6B, Tb¹ (broad expression),

P{w+mW.hs=GawB}c204/TM3 (follicle cells), Ser¹;

P{w+mW.hs=GawB}c323a (follicle cells) P1 {w+mW.hs=GawB}OK107 (adult brain, mushroom bodies), CyO; and,

P{ry+t7.2=1ArB}A350.1M2/b1 Adh*cn* 1(2)**;ry⁵⁰⁶ (CNS and follicle cells).

A Gal4 driver is a member of a transgenic fly line. Gal4 relates to the Gal4 gene, which encodes a transcriptional activator. A promoter (or enhancer) directs expression of the yeast transcriptional activator GAL4 in a particular pattern, and GAL4, in turn, directs transcription of the GAL4-responsive (UAS) target gene in an identical pattern. The system's key feature is that the GAL4 gene and UAS-target gene are initially separated into two distinct transgenic lines. In the GAL4 line, the activator protein is present, but has no target gene to activate. In the UAS-target gene line, the target gene is silent because the activator is absent. It is only when the GAL4 line is crossed to the UAS-target gene line that the target gene is turned on in the progeny (Brand A H, Perrimon N. Targeted gene expression as a means of altering cell fates and generating dominant phenotypes. Development June 1993; 118(2):401-15).

The GAL4 system is a method for directed gene expression that allows genes to be expressed ectopically in numerous cell- or tissue-specific patterns. The technique is exploited to study Drosophila melanogaster all stages of development, from the embryo to the adult.

For X-linked insertions, recombinants with a yw chromosome were generated, and all stocks were introgressed into a yw;pol background to facilitate nondisjunction tests. Unless noted otherwise, the wild type stock referred to herein refers to yw; Pw⁺ {nos.GAL4::VP16}/+;pol.

### Example 3

Fixations and antibody labeling of late state oocytes were performed according to Matthies and Hawley, (1999). Earlier meiotic stages were fixed under the same buffer conditions. However, these fixations were done in the presence of 5% formaldehyde (Ted Pella) for 20 minutes. Cyanine or Alexa Fluor-conjugated secondary antibodies were used according to the manufacturer (Jackson Immunoresearch; Molecular Probes).

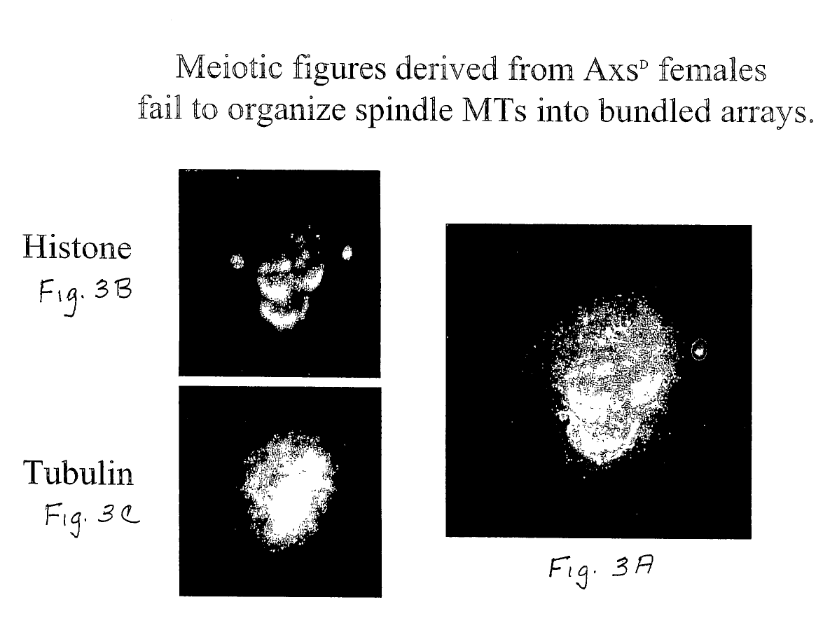

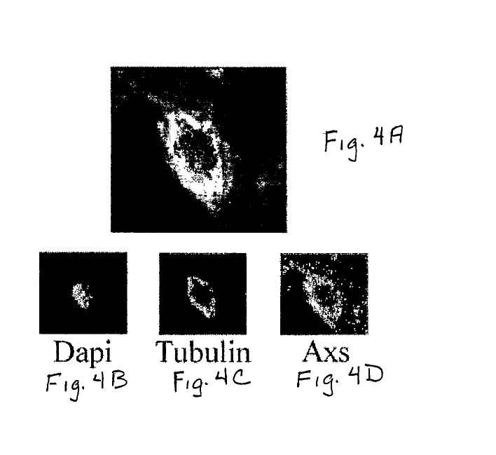

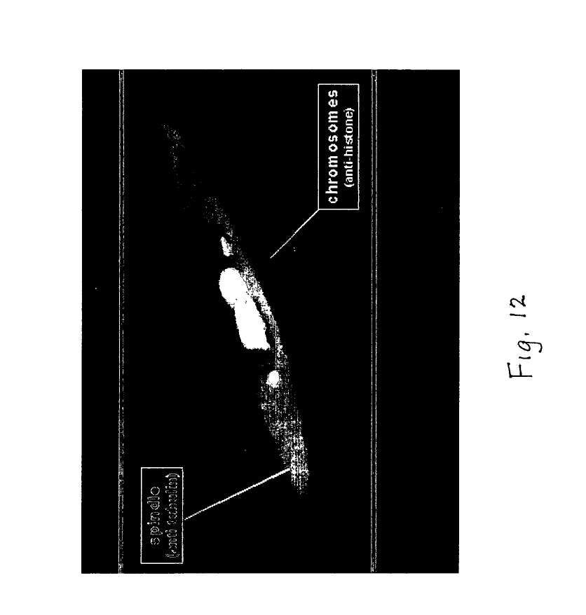

The method utilized for colchcine treatment and subsequent fixation of embryos is described in Stevenson et al., 2000. Primary antibody dilutions were as follows: Mouse anti-lamin antibodies were used at 1/100 (ADL84; Paul Fisher), rat anti-tubulin 1/250 (Chemicon, Inc.), rat anti-tubulin (Chemicon, Inc), and rabbit anti-axs 1/100. Tubulin is monomeric subunit of the microtubule cytoskeleton. Colchicine binds to tubulin and prevents the addition of subunits to lengthen the microtubule. FIGS. 3, 4, 6, 7, 11, 12, and 13 illustrate photomicrographs wherein the above antibodies were used. FIG. 3 shows failure to organize spindles in Axs^{D} females.

### Example 4

Data for micrographs were obtained using an inverted Olympus scope fitted with a cooled CCD for deconvolution analysis. Data were collected in 0.25 micron Z steps 4-10 microns above and below the element of interest. The resulting data were subjected to deconvolution using the Software package (API).

### Example 5

The Axs mutation was mapped genetically between rudimentary (r) and forked 69 [Whyte, 1993 #1], with the mapping experiments indicating that the Axs mutation maps approximately 14 kb to the right of r. The Axs mutation had been shown to be partially complemented by a duplication Dp(1:4)r^{+7c} (14B13; 15A9). To further refine its position, a series of Dp(1;4)r^{+75c} deletion derivatives were tested that had breakpoints in 14F-15A, none of which covered Axs. Most notably, the derivative Dp(1:4)80 g8d which breaks approximately 10 Kb proximal to r failed to cover Axs. Hence, it was concluded that at least a part of the Axs gene is within 10 kb proximal to r. This is illustrated in FIGS. 1a and 1b.

Next, a set of overlapping fragments (SCX1, SCR2, and SCX2) were subcloned that spanned the 30 kb region proximal to r into a P element transformation vector and obtained germline transformants, as shown in FIG. 1a. These transgenes were tested for their ability to rescue the meiotic nondisjunction phenotype of Axs^{r2}/(1)dl-49,Axs^{r2} females. As can be seen, below, the meiotic nondisjunction was rescued.

TABLE 1
ABILITY OF P ELEMENT RESCUE CONSTRUCTS TO RESCUE THE
MEIOTIC PHENOTYPE IN AXS^{r2}DL-49, AXs^{r2} Females

| CON- | LINE | CONTROL | | | P ELEMENT | | |
|---|---|---|---|---|---|---|---|
| STRUCT | NO. | X ND** | 4 ND*** | N* | X ND | 4 ND | N |
| SCX1 | 12 | 14.3 | 3.1 | 298 | 0.0 | 0.2 | 643 |
| | 13 | 17.5 | 5.6 | 491 | 0.9 | 1.6 | 665 |
| SCX2 | 40 | 13.0 | 6.6 | 173 | 2.2 | 0.0 | 540 |
| SCR2 | 52 | 9.0 | 2.1 | 401 | 17.0 | 4.3 | 635 |
| | 57 | 8.3 | 1.3 | 300 | 8.9 | 1.6 | 537 |
| | 58 | 11.2 | 2.4 | 589 | 13.0 | 2.0 | 459 |

*N = the number of members tested.
**X ND = chromosome X crossed for nondisjunction.
***4 ND = chromosome 4 crossed for nondisjunction.

These experiments restricted the location of the Axs locus to an 11 kb region representing the overlap between constructs SCX1 and SCX2. Next, additional fragments were constructed and tested which localized Axs to a five kb region in the construct XS1, shown in FIG. 1b.

Two potential transcription units were apparent within this five kb region. One corresponds to the 3′ end of a septin-related gene designated CG9699 by the Berkeley Drosophila Genome Project. Rescue constructs covering the complete septin gene (SCR2) did not appear to rescue the chromosome segregation defects associated with the Axs^{r2} allele. The other represents a novel protein, which, according to details given below, appears to be Axs.

Nondisjunction tests performed in homozygous Axs^{r2} females carrying a copy of the genomic transgene SCX1 rescued the segregation defects associated with this recessive allele, and Axs^{D} females carrying a copy of the XS1 transgene were partially rescued.

### Example 6

Next, an ovarian cDNA library of Drosophila was screened with a genomic fragment spanning the Axs gene and a single cDNA was obtained whose sequence was completely contained within the XS1 transgene. This partial cDNA was polyadenylated and contained an open reading frame of 1938 nucleotides encoding a predicted 646 amino acid protein. The Axs gene corresponds to BDGP transcription unit CG9703. Full-length cDNAs were isolated as a part of the genome sequencing project (EST clone LP10412) and contained approximately 500 nucleotides of nontranslated 5′ leader sequence.

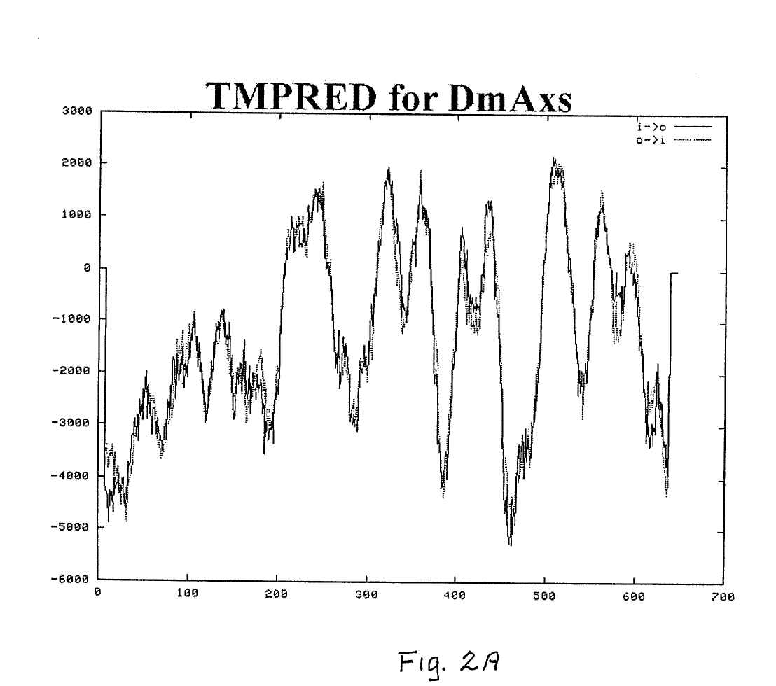

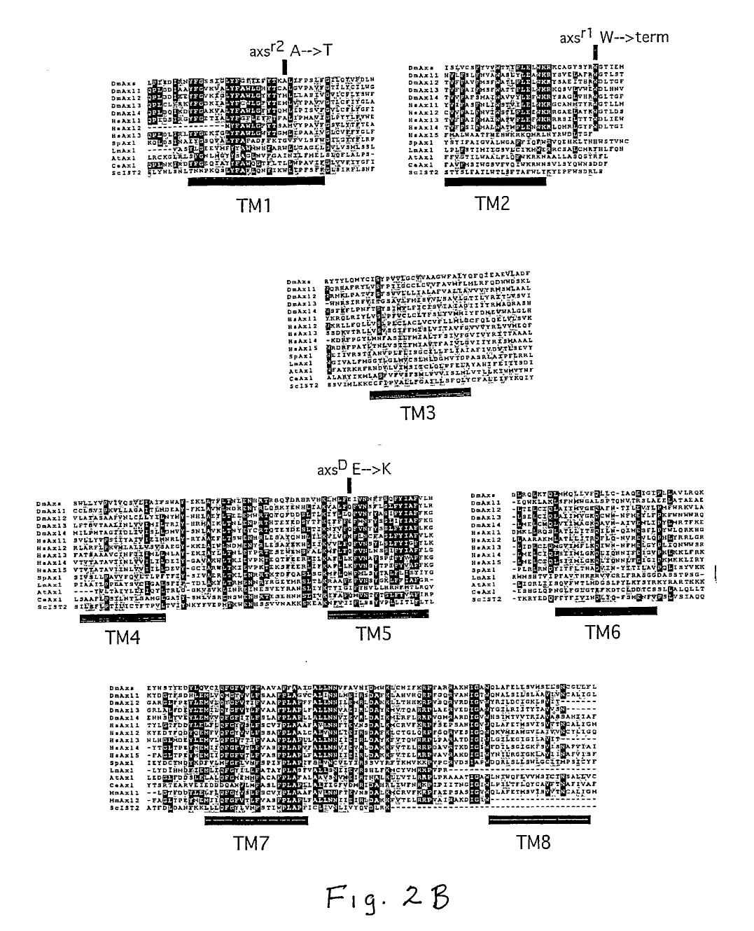

It was determined that Axs defines a new gene family of predicted transmembrane proteins. The predicted translation product encoded a protein of 646 amino acids with a molecular weight of 75 kD. The Axs polypeptide was unusually rich in hydrophobic amino acids (LVIFMW=37.5%), and any one of several transmembrane prediction algorithms predicted the existence of 7-8 transmembrane segments spaced 35-60 amino acids apart, as shown in FIG. 2a. A hydrophobicity is shown in FIG. 2a. The predicted transmembranes are further shown in FIG. 2b. This was determined using the Tmpred program. This program makes predicting of membrane-spanning regions and their orientation. The algorithm is based on the statistical analysis of Tmbase, a database of naturally occurring transmembrane proteins(Hofmann, K., & Stoffel, W. TMbase—A database of membrane spanning proteins segments. Biol. Chem. Hoppe-Seyler, 374,166 (1993).). Comparison of the primary sequence to the current databases indicates that the Axs protein defines a new family of transmembrane proteins with one or more representatives in eukaryotes for which significant genomic studies have been undertaken. (expand) These are all characterized by a conserved transmembrane core containing eight transmembrane domains (FIG. 2). No significant homology was observed outside of the transmembrane segments possibly indicating diverse ligand and signaling potentials among different family members.

After isolation of the Axs gene, it was desired to determine the composition of the Axs^{D} gene. The molecular lesions for the original Axs^{D} allele and each of the three existing intragenic suppressors of Axs^{D} are shown at FIG. 2b and FIG. 7 (Whyte et al., 1992). The Axs^{D} mutation was induced by a G→A transition at position 1653 of the full-length cDNA and resulted in the exchange of a glutamic acid residue for a lysine residue at amino acid 400 of the primary sequence. Notably, this change occurred within a highly conserved predicted transmembrane region of the protein (FIG. 2b). As expected, it was observed that the three intragenic suppressor alleles of Axs all possess this same mutation. In addition, each exhibited another single nucleotide change located elsewhere in the transcription unit. Axs^{r1} exhibits a G→A transition at position 1264 resulting in the introduction of a stop codon at amino acid 270 and Axs^{r2} is induced by a G→A transition at position 1122 eliciting a change from alanine to threonine for amino acid 223. The Axs^{r3} allele showed no changes in the coding sequence when compared to its parent chromosome. However, a single nucleotide change was observed in the untranslated 5′ leader and it is likely that the mutations' ability to suppress the effects of the Axs^{D} mutation is regulatory in nature.

Thus, the above information determined the sequence of the nucleotide sequences for Axs and the mutant alleles.

### Example 7

A GAL4/UAS fly system was used to determine the tissue in which Axs^{D} exerted its effects on chromosome segregation. Expression of both the wild-type Axs⁺ and Axs^{D} allelic forms of the Axs protein in either the germline (nanos. GAL4) or follicular epithelium of the Drosophila ovary was directed.

The maturing oocytes are surrounded by an epithelial layer, producing a structure called the follicle. Ooctyes surrounded by a flattened, single, layer of epithelium are the primordial follicles (Primordial Follicles). As the primordial follicles begin to grow, the surrounding follicular epithelium (or granulosa cells) change morphology. Egg chambers offer an excellent system for cell biological analysis because they have a relatively simple architecture, comprised of large germ cells surrounded by a monolayer follicular epithelium. Further, the coordinated maturation of germ cells with the follicular epithelium provides an excellent system to study the synchronous development of two distinct tissue types. Study of both the germline and follicular epithelia allowed for analysis of the effects of Axs.

GAL4 lines directing expression in the adult brain, central nervous system (CNS), and weak, broadly expressing drivers such as ACT5C-GAL4 failed to elicit any effect on fertility or chromosome segregation (data not shown). In contrast, GAL4 lines driving expression of the Axs^{D} protein in either the germline (nanos.GAL4) or follicular epithelium of the Drosophila ovary resulted in a severe reduction in female fertility. Fertility and meiotic chromosome segregation were monitored in females expressing either the Axs⁺ or Axs^{D} protein under the control of various cell- and stage-specific GAL4 drivers. This effect was allele specific and occurred whether the protein was expressed in either the germline or somatic components of Drosophila ovaries. In contrast, expression of the Axs+protein has no discernable affect upon fertility when expressed in either the somatic or germline components of the Drosophila ovary. The results are listed below in Table 2:

TABLE 2
OVARIAN EXPRESSION OF AXS AND AXS^{D} REDUCES FEMALE FERTILITY
AND DISRUPTS ACHIASMATE CHROMOSOME SEGREGATION

| | | | | |
|---|---|---|---|---|
| | Hatching | Adj. | | 4th |
| GENOTYPE | Rate | Total | XNDJ | NDJ |

Germline Expression

| | | | | | |
|---|---|---|---|---|---|
| 1. | yw/yw, UAS-Axs^{D}#1/+; GAL4::VP16-nos. UTR/+, pol | 15% | 1473 | 4.1 | 32.4% |
| | | N = 658 | | | |
| 2. | FM7w/yw, UAS-Axs^{D}#1/+; GAL4::VP16-nos. UTR/+; pol | 12% | 1530 | 41.4% | 40.3% |
| | | N = 1077 | | | |
| 3. | FM7w/yw; GAL4::VP16-nos. UTR/UAS-Axs^{D}#2; pol | 9% | 3093 | 44.3% | 36.0% |
| | | N = 1481 | | | |
| 4. | FM7w/yw; GAL4::VP16-nos. UTR/+; pol | 92% | 2225 | <1% | <1% |
| | | N = 498 | | | |
| 5. | FM7w/yw; UAS-Axs^{D}#2/+; pol | 86% | 3724 | <1% | <1% |
| | | N = 640 | | | |
| 6. | FM7w/yw; UAS-Axs^{D}#3/+; GAL4::VP16-nos. UTR/UAS-Axs^{D}#2; pol | 7% | 607 | 42.7% | 34.0% |
| | | N = 2002 | | | |
| 7. | FM7w/yw; GAL4::VP16-nos. UTR/UAS-Axs^{D}#2; pol | 88% | 2265 | <1% | <1% |
| | | N = 382 | | | |
| 8. | FM7w/yw; UAS-Axs^{wt}#1; GAL4::VP16-nos. UTR/UAS-Axs^{wt}#2; ;pol | n.d. | 4047 | <1% | <1% |
| 9. | yw, UAS-Axs^{wt}#1; GAL4::VP16-nos. UTR/UAS-Axs^{wt}#2; pol | n.d. | 2228 | <1% | <1% |
| 10. | FM7w/yw; UAS-Axs^{wt}#1; UAS-Axs^{D}#1/+; GAL4::VP16-nos. UTR/+; pol | n.d. | 772 | 42.0% | 32.8% |

Follicular Expression

| | | | | | |
|---|---|---|---|---|---|
| 11. | FM7w/yw; FollicleGal4#2^{P(w + mW.hs = GawB)c2o4}/UAS-Axs^{D}; pol | 8% | 832 | <1% | <1% |
| | | N = 1365 | | | |
| 12. | FM7w/yw; FollicleGal4#1^{(w + mW hs = GawB)c323a}; /UAS-Axs^{D}#1 + ; pol | 4% | 758 | <1% | <1% |
| | | N = 1763 | | | |
| 13. | FM7w/yw; FollicleGal4#2^{P(w + mW hs = GawB)c2O4}/UAS-Axs^{wt}#2; pol | 84% | 1369 | <1% | <1% |
| | | N = 444 | | | |

Consistent with previous reports using duplications, increasing the dosage of wild-type Axs had no discernable effect on chromosome segregation or female fertility when expressed in either the germline or follicle cells, as shown in Rows 7, 8, 9, and 13, Table 2. Even in the presence of two or more copies of the UAS-Axs⁺ transgene, achiasmate chromosome segregation proceeded normally. In general, expression of the Axs^{D} protein in the ovary had dramatic effects on female fertility, as shown in the remaining rows of Table 2. Expression of the dominant allele of the Axs protein resulted in reduced fecundity and high levels of achiasmate chromosome nondisjunction when expressed in the germline. In contrast, expression of this same construct in follicle cells resulted in reduced fecundity; however, gametes derived from these females exhibited no discernable defects in chromosome segregation (Table 2). This data is further summarized in FIG. 9.

Gametes derived from females expressing the Axs^{D} allele exhibited segregation defects similar to that reported for the original Axs^{D} allele, with the overall frequency of X chromosome segregation similar to that originally reported by Whyte et al. (1988). The defects produced under these conditions were similar in overall frequency and character to those seen with the original Axs^{D} mutation (Zitron and Hawley, 1989). Moreover, the effects were achiasmate-specific and simultaneous XX←→44 segregations predominated in gametes exhibiting chromosomal nondisjunction. These data restrict Axs role in chromosome segregation to the Drosophila germline.

Axs′ effects on chromosome segregation are allele specific. Expression of this protein in the somatic follicle cells failed to produce this effect, and thus, the dominant allele of Axs, Axs^{D}, exerted its effects in the germline component of the Drosophila ovary. Also, a comparison of segregation defects associated with various genes is shown at FIG. 8. It is shown that achiasmate segregation defects in Axs are similar to P40 and P21.

### Example 8

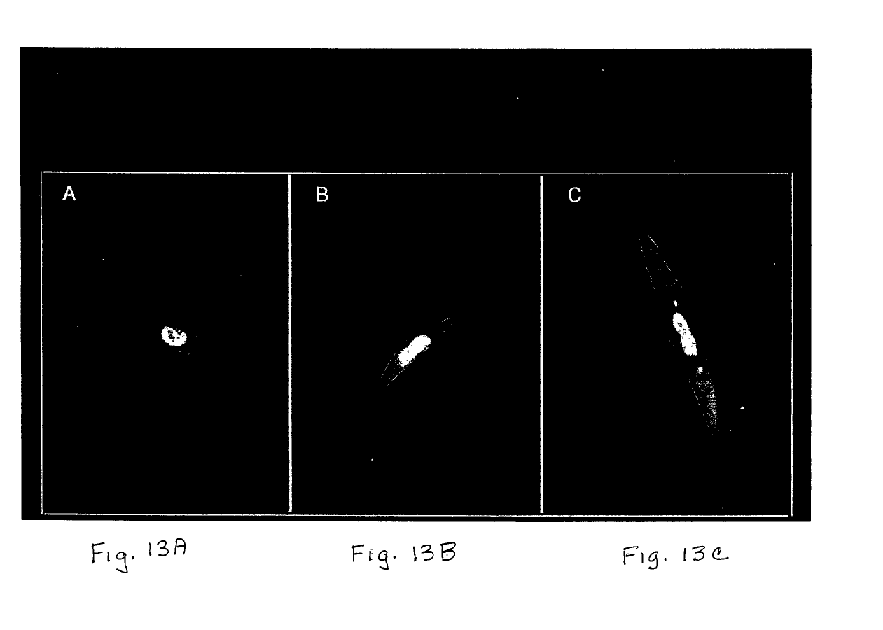

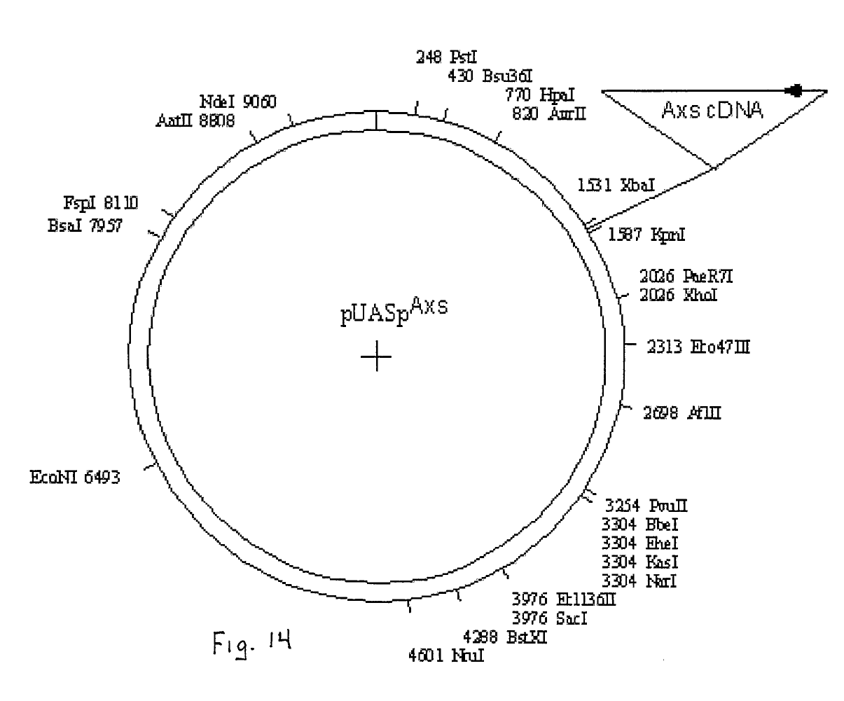

The meiotic figures of oocytes derived from Axs^{D} females were examined in order to determine the nature of the defect evident from the genetic studies presented above. Toward this end, both wild type and Axs^{D} expression stage oocytes were labeled with tubulin and DNA probes. Labeling was done according to known procedures. As was observed, germline cysts expressing Axs^{D} appeared normal with respect to their overall morphology. Nurse cell/oocyte number and positioning were uncompromised, and the distribution of patterning (e.g., grk) and cytoskeletal components (e.g., tubulin, actin) appeared to be unaffected (data not shown). Gross defects only became apparent in late stage post-vitellogenic oocytes (FIG. 3). In these Axs^{D}—expressing oocytes, meiotic spindle assembly failed to occur properly. Normal assembly and segregation is shown at FIGS. 12 and 13.

Analyses of fixed samples indicate that Axs^{D}—expressing oocytes failed to assemble bipolar spindles, as shown in FIG. 3. The karyosomes present in these meiotic figures were typically broken and the chromosomes were “individuated” and condensed. This configuration is indicative of entry into meiotic metaphase I (Page AW, Orr-Weaver TL. Activation of the meiotic divisions in Drosophila oocytes. Dev Biol Mar. 15, 1997;183(2):195-207). However, only a small fraction of these oocytes exhibited any indication of the microtubule bundling and bipolarity typically achieved in wild type meiotic figures. Instead, microtubules were diffusely associated with the condensed chromatin and failed to develop distinct poles as assessed by the spindle pole antigen d-TACC. Typical bipolar spindles were rarely observed in these oocytes (2.2%; N=148) whereas the control figures almost always achieved bipolarity (84%; N=126). When compared to wild-type figures similarly prepared, it was evident that in mutant oocytes, the process of bundling microtubules into a bipolar structure was defective. Tubulin staining was observed surrounding each of the individualized meiotic chromosomes, but the formation of a tapered bipolar spindle was rarely observed (FIG. 3). This is further shown at FIG. 13. This is a comparison between normal assembly and failed assembly.

The microtubule labeling observed in mutant oocytes is reminiscent of early spindle assembly events occurring in wild-type oocytes. Early spindle assembly has been reported to initiate from the meiotic chromosomes with microtubules assembling upon the nascent karyosome and eventually being bundled and sculpted into a bipolar spindle. During this process, individual chromosome pairs (bivalents in the case of achiasmate chromosomes) became apparent and were arrayed upon the bipolar spindle in stereotyped fashion (Matthies and Page Orr-weaver). Although spindle assembly appears to be compromised in Axs^{D}—expressing oocytes, the chromosomes were discrete and individualized (FIGS. 3 and 13).

Female Drosophila meiosis is anastral, and the chromosomes are believed to be the central organizing elements in the assembly of the spindle. Early spindle assembly is characterized by the appearance of microtubules on the surface of the karyosomal chromatin. Concomitant with breakdown of the karysome and positioning of the bivalents, these microtubules are eventually sculpted into a tapered bipolar spindle.

In accordance with this observation, the localization of the spindle pole antigen, d-TACC was disrupted in these figures as well. d-TACC normally weakly labels the spindle and is specifically enriched at the anastral poles (RAFF and MSPS paper). No such enrichment is evident in Axs^{D} oocytes. Instead, d-TACC uniformly co-localized with the diffuse tubulin labeling surrounding each of the chromosomes and showed no enrichment at points distal from the chromosomes.

### Example 9

It was determined that Axs localized to cellular membranes and microtubules during meiosis. In order to determine the subcellular localization of Axs, polyclonal antibodies were raised against the relatively non-conserved amino terminal region of the Axs protein (See Example 3). Affinity-purified fractions prepared against the immunogen recognized a 75 kD protein as well as a number of putative degradation products in ovaries over-expressing the Axs⁺ protein. The preparation also recognized similarly sized products driven from a UAS-Axs^{+MYC} transgene that are also recognized by a myc antibody. These products were dependent upon full reconstitution of the UAS Axs⁺/nos-GAL4 system (FIG. 8). Axs protein products were not detected in matched samples prepared from wild-type ovaries possibly indicating that the native product was rare.

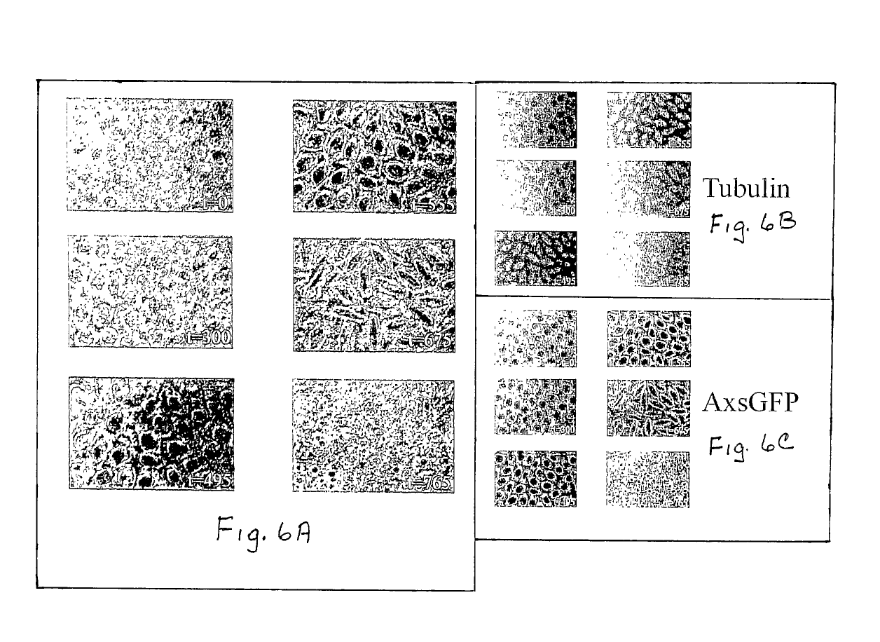

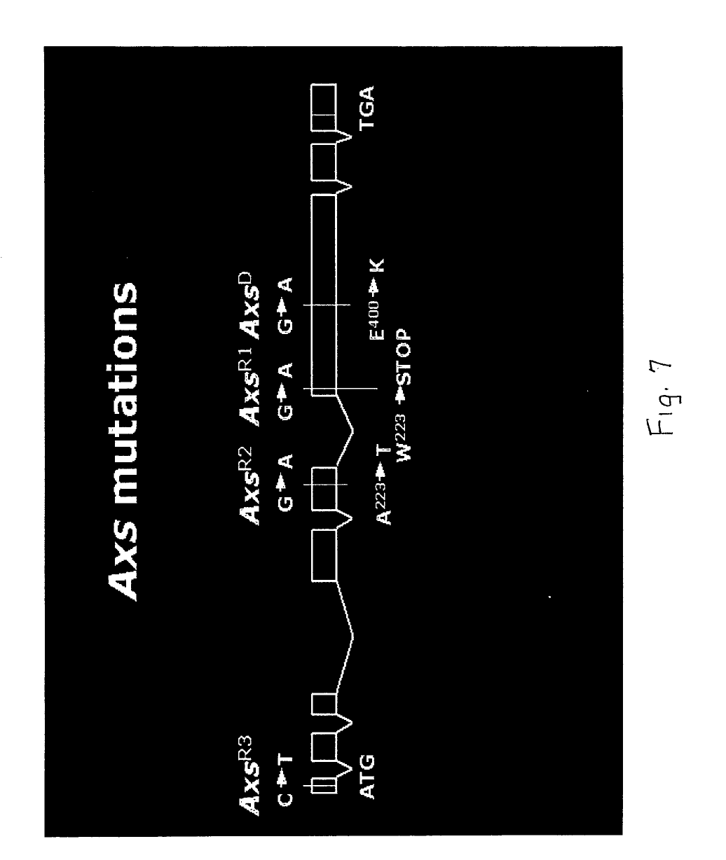

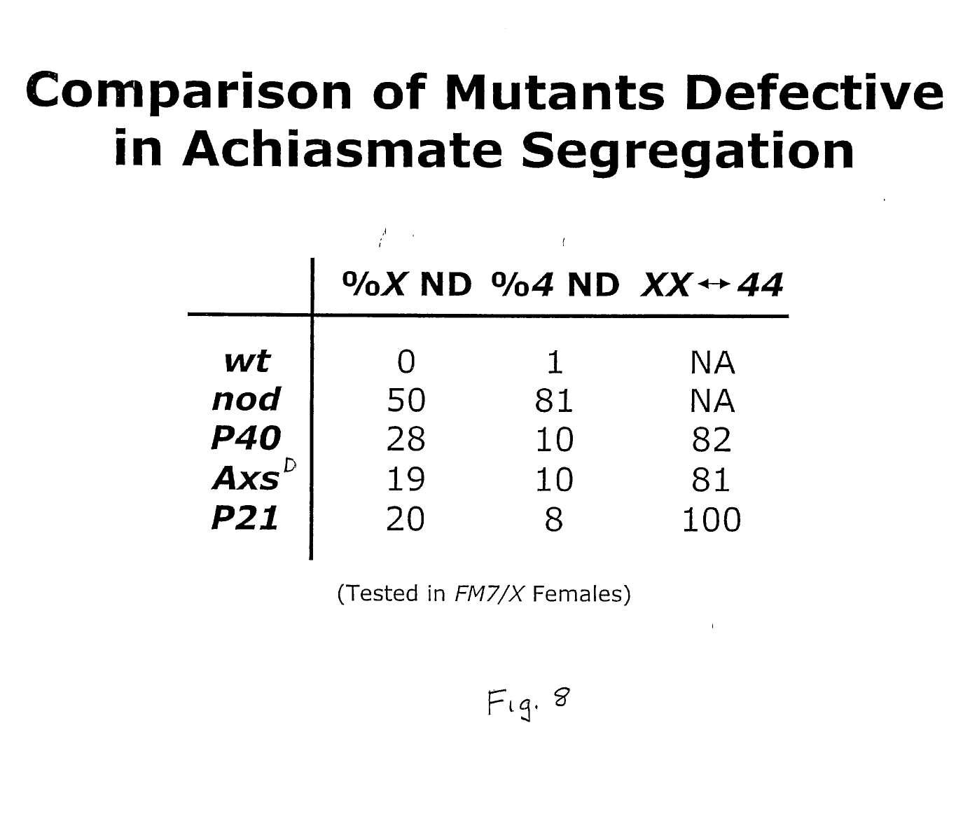

In stage 5 egg chambers, it was observed that in wild-type oocytes, the Axs protein was present in the germline component of developing stage 5 egg chambers and was localized to large occlusions in both the nurse cells and oocyte (FIG. 6). The Axs-containing bodies within the oocyte appeared to be associated with the oocyte nuclear membrane and were closely opposed to lamin distribution. This labeling was much more prominent in embryos over expressing the Axs⁺ or Axs^{+MYC} protein as was an increase in nurse cell nuclear membrane labeling (FIG. 6, data not shown).

Axs protein was also found to be associated with microtubules. During oogenesis, Axs protein distribution closely followed the distribution of bulk microtubules. In early oogenesis, an MTOC organized microtubules in the posterior of the oocyte. The protein was present at the cortex (FIG. 6) in the form of small occlusions or vesicles, which were associated with microtubule containing bundles. Similar vesicles were also seen associated with the spindle and nuclear cell surface of meiotic or mitotic figures, respectively (FIG. 6).

During the vitellogenic stages of oogenesis, the Axs protein remained associated with the oocyte nucleus but also appeared at the cell surface in association with cortical microtubules. Axs-containing particles co-localized with microtubule bundles present at the nurse cell and oocyte cell surface (FIG. 6). Similar particles were seen coalesced around the oocyte karyosome during the early stages of spindle assembly and ensheathing of the mature bipolar meiosis I spindle (FIG. 6).

It was determined that the Axs protein was associated with early meiotic figures and ensheathed mature meiotic spindles. It was noted that the presence of Axs protein on the assembling spindle was roughly coincident with the defects observed in oocytes expressing the Axs^{D} protein.

During the first meiotic division, mature meiotic figures were not bounded by either lamin or nuclear envelope antigens (data not shown). Axs, however, localized to a sheath that appeared to encapsulate the forming meiotic spindle. Moreover, appearance of this sheath closely approximated the degree to which spindle assembly has progressed, with mature spindles exhibiting a tight association with the spindle surface (FIGS. 6). The nature of this sheath is unknown. It is unclear whether the Axs protein present in this structure is a component of a continuous bilayer, or is present as a series of vesicles, which accumulate upon microtubule bundles comprising the spindle.

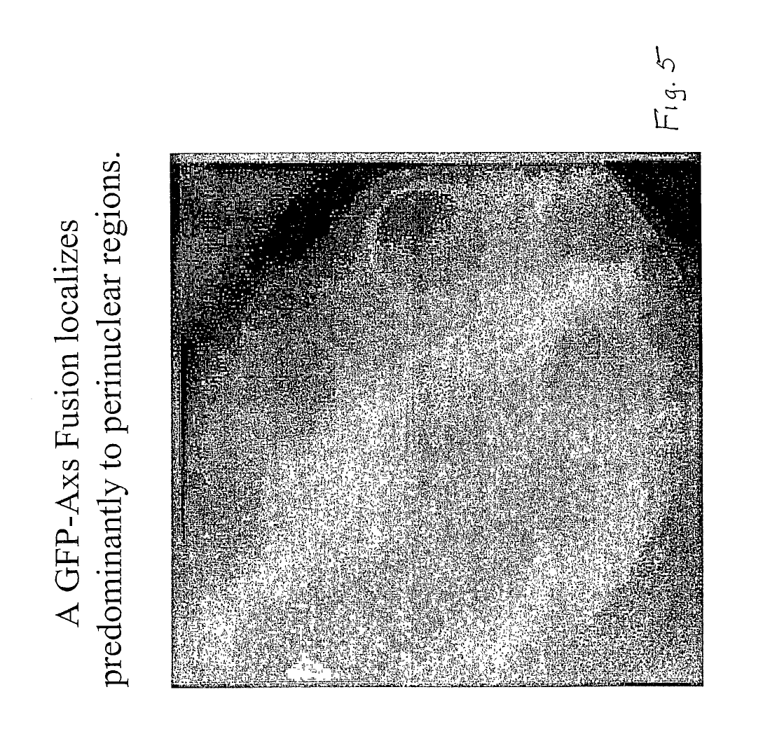

Antibodies raised against the Axs protein were used, as well as epitope tagged versions to determine the subcellular localization of the Axs protein. Consistent with its primary sequence, the protein appeared to localize to the cell membranes in Drosophila ovaries and embryos (FIGS. 6 and 5). In both germline cysts and early synticial embryos, the protein was both diffusely cytoplasmic and present upon the nuclear membrane. Interestingly, the protein appeared to be redistributed as a function of cell cycle state. In the early embryo, live imaging of an Axs⁺ protein indicated that the protein cycles between the cytoplasm and nuclear surface during interphase and metaphase, respectively (FIG. 6). This is similar to the pattern observed upon meiotic figures. Axs protein is associated with early meiotic figures and ensheaths mature meiotic spindles. The presence of Axs protein on the assembling spindle was roughly coincident with the defects observed in the oocytes expressing Axs^{D} protein.

Experiments performed on early embryos suggest that depolymerization of microtubules results in a redistribution of the protein while leaving nuclear integrity intact (FIG. 6). Thus, regardless of the phase of the protein, bilayer or vesicular, its mitotic and meiotic localization appears to be dependent upon microtubules.

### Example 10

It was determined that Axs localization is cell cycle and microtubule dependent in mitotic cells as well. The early synticial divisions of the Drosophila embryo were used to assess the role of microtubules in Axs localization. A UAS-Axs^{+GFP} transgene was constructed to permit live imaging. The AxsGFP fusion was expressed during oogenesis in a pattern similar to that described above for the Axs⁺ protein. In addition, the synticial divisions of embryos expressing the transgene were imaged. FIG. 6 shows a time course corresponding to a complete cell cycle of an Axs^{+GFP} embryo injected with Rhodamine tubulin. AxsGFP was observed to cycle to and from the nuclear membrane as a function of cell cycle state. During interphase, the protein was dispersed and cytoplasmic (FIG. 6). As spindle assembly proceeded, starting at prometaphase and extending through telophase, the protein became tightly localized to the juxtaspindle region nuclear cell surface with prominent accumulations bracketing the spindle poles (FIG. 6). During telophase the protein associated with the midbody and the daughter products' nuclear cell surface (FIG. 6).

In order to determine whether Axs localization was dependent upon microtubules, a fixation protocol was used that allowed for the exposure to the microtubule depolymerizing agent, colchicine (Stevenson et al., 2000). Examination of fixed mock samples suggested localization of Axs⁺ protein occurs coincident with spindle assembly. Axs protein expressed from the UAS-Axs+transgene localized to the nuclear cell surface as the chromosomes began to condense and the MTOCs became active (FIG. 6). During metaphase, it was enriched near the spindle poles in a broad band extending towards the metaphase plate (FIG. 6).

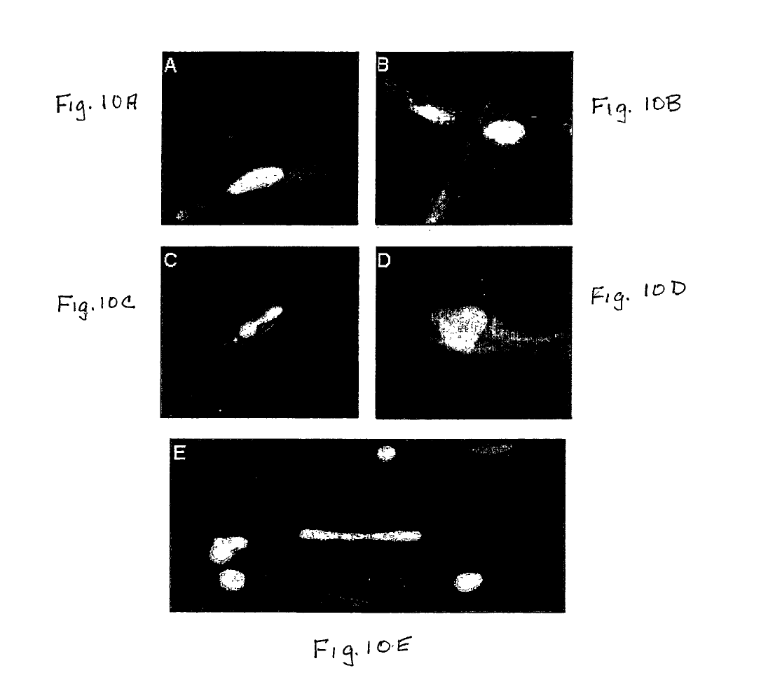

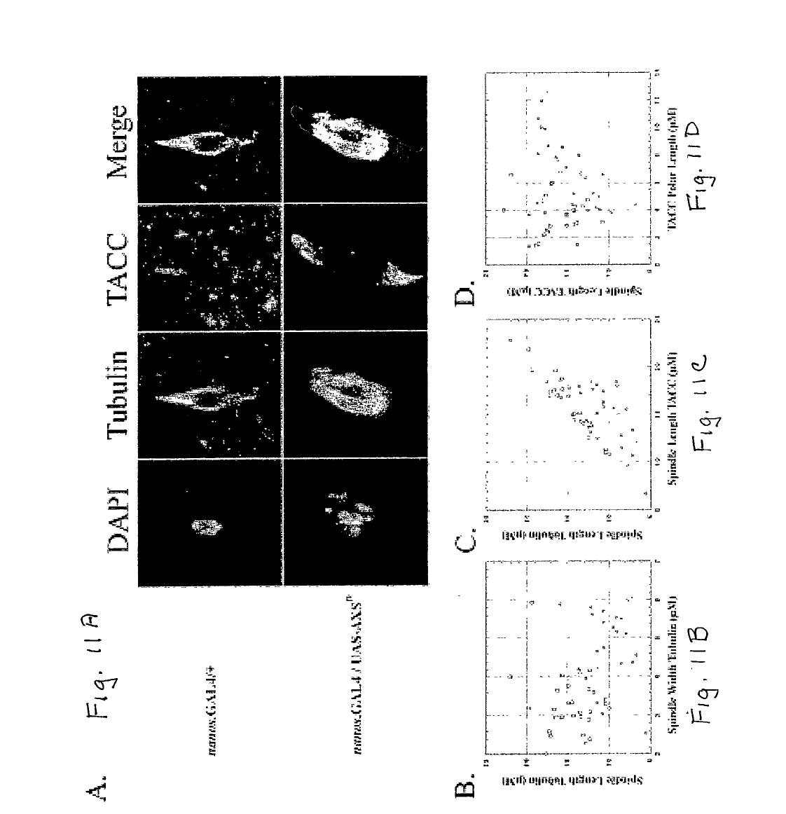

Embryos were exposed to levels of colchines sufficient to prevent spindle function and detectable MTOC activity (FIG. 10, data not shown). As a result, the nuclei exhibited condensed metaphase chromosomes, which were unable to align in the absence of MTOC function (FIG. 10). Exposure times as little as 2 minutes produced similar effects. In the absence of MTOC function, the Axs protein became relatively disorganized. The protein still localized to the nuclear surface. However, it never achieved the half-hemisphere band pattern seen in mock treated embryos (FIG. 10. In addition, large aggregates of the protein were distributed on the nuclear surface. These aggregates did not appear to coincide with the inactive spindle poles as evidenced by gamma tubulin labeling (data not shown).

The synticial cell divisions characteristic of early Drosophila embryogenesis represent a somewhat specialized form of mitosis. Nuclear lamina and their associated membranes never completely break down until mitosis during these abbreviated cell cycles (Paddy et al., 1996), and nuclear divisions are accomplished through a “closed” mitosis where the spindle remains circumscribed by both lamin and nuclear envelope until the chromosomes begin to undergo anaphase movement (Paddy et al.). The mitotic localization of the Axs protein closely reflects the polarity of the spindle poles, with prominent accumulations of the protein occurring at or near the MTOC (FIGS. 6 and 10). Moreover, depolymerization of microtubules during these mitoses results in the mislocalization of Axs protein. Upon treatment with colchcine, the Axs protein no longer exhibited an association with the spindle poles, and was distributed more uniformly upon the nuclear surface. Under these conditions, lamin and nuclear envelope antigen localization remains unaffected. Also, large occlusions were formed along the nuclear surface, which did not appear to be localized with any clear relationship to the inactive spindle poles.

### Example 11

A method for predicting spindle formation during female meiosis can be practiced, whereby the method is initiated by isolating an Axs^{D} gene. The gene is found herein as SEQ. ID NO. 1. The gene will be isolated according to known procedures. Once isolated, the gene can be used to form a cDNA probe. The probe will be formed from the isolated nucleic acid molecule that formed the Axs^{D} gene. Additionally, the probe will be labeled according to known procedures. The gene probe will then be contacted with DNA from a homolog. Attachment of the gene probe will indicate, in a female, the presence of a mutant gene analog. This indicates a high likelihood that spindle formation will not occur during female meiosis I, and nondisjunction will result.

### Example 12

A method for predicting spindle formation during female meiosis can be practiced, whereby the method is initiated by isolating an Axs gene. The gene is found herein as SEQ. ID NO. 1. The gene will be isolated according to known procedures. Once isolated, the gene can be used to form a cDNA probe. The probe will be formed from the isolated nucleic acid molecule that formed the Axs gene. Additionally, the probe will be labeled according to known procedures. The gene probe will then be contacted with DNA from a homolog. Attachment of the gene probe will indicate, in a female, the presence of a mutant gene analog. This indicates a high likelihood that spindle formation will not occur during female meiosis I, and disjunction will result.

### Example 13

A method can be practiced for destroying a defective oocyte, whereby nondisjunction occurs during meiosis. The method is initiated by isolating an Axs^{D} gene. The gene is listed herein as SEQ. ID NO. 1. The gene will be isolated according to known procedures. Once isolated, the gene can be used to form a cDNA probe. The probe will be formed from the isolated nucleic acid molecule that formed the Axs^{D} gene. Additionally, the probe will be labeled according to known procedures. The gene probe will then be contacted with DNA from a homolog. Attachment of the gene probe will indicate, in a female, the presence of a mutant gene analog. This indicates a high likelihood that spindle formation will not occur during female meiosis I, and nondisjunction will result. Once a mutant is identified, the oocyte can be destroyed according to known procedures.

### Example 14

A method can be practiced for rescuing a defective oocyte, whereby nondisjunction occurs during meiosis. The method is initiated by isolating an Axs^{D} gene. The gene is listed herein and is SEQ. ID NO. 1. The gene will be isolated according to known procedures. Once isolated, the gene can be used to form a cDNA probe. The probe will be formed from the isolated nucleic acid molecule that formed the Axs^{D} gene. Additionally, the probe will be labeled according to known procedures. The gene probe will then be contacted with DNA from a homolog. Attachment of the gene probe will indicate, in a female, the presence of a mutant gene analog. This indicates a high likelihood that spindle formation will not occur during female meiosis I, and nondisjunction will result. Once a mutant is identified, the oocyte can be rescued by contacting the oocyte with an Axs protein during meiosis, whereby this will cause disjunction to occur.

### Example 15

The Axs protein may be expressed in a transgenic animal whose germ cells contain a gene, which encodes the Axs protein and which is operably linked to a promoter effective for expression of the Axs gene. The transgenic animal can be prepared from a variety of non-human animals, including mice, rats, rabbits, sheep, dogs, goats, and pigs (see Hammer et al., Nature 315:680-683, 1985, Palmiter et al., Science 222:809-814, 1983, Brinster et al., Proc. Natl. Acad. Sci. USA 82:4438-4442, 1985, Palmiter and Brinster, Cell 41:343-345, 1985, and U.S. Pat. Nos. 5,175,383, 5,087,571, 4,736,866, 5,387,742, 5,347,075, 5,221,778, and 5,175,384).

An expression vector, including the nucleic acid molecule to be expressed, together with appropriately positioned expression controlled sequences, is introduced into the pronuclei of an egg, for example, by micro-injection. Integration of the injected DNA is detected by blot analysis of DNA from tissue samples. It is possible to have tissue-specific expression by relying on the use of a tissue-specific promoter or an inducible promoter, which will allow regulated expression of the transgene.

The Axs protein can then be isolated by, among other methods, culturing suitable host and vector systems to produce the recombinant translation products of the invention. Supernatants from such cell lines or protein inclusions or host cells, where the Axs protein is not excreted into the supernatant can then be treated by a variety of purification procedures, in order to isolate the desired Axs protein. For example, the supernatant may be first concentrated using commercially available protein concentration filters, such as an Amicon or Millipore Pellicon Ultra-filtration unit. Following concentration, the concentrate can be applied to a suitable purification matrix, such as an anti-Axs protein antibody bound to a suitable support. Alternatively, anion or cation exchange resins may be employed in order to purify the Axs protein. A further alternative is the use of one or more reverse phase, high performance, liquid chromatography (RP-HPLC) steps, which may be employed to further purify the Axs protein. Other methods of isolating Axs proteins are well known in the skill of the art.

### Example 16

Another transgenic animal example would include a transgenic animal, which lacks the Axs gene (for example, a knock-out mouse). This knock-out animal would be prepared in the same way as the above example indicates for preparation of a transgenic animal.

The complete sequence of the mouse Axs gene and its 5′ to 3′ flanking regions could be determined. Once the mouse Axs gene is determined, a fragment containing the entire gene body, plus a 5′ flanking sequence and a 3′ flanking sequence could be sub-cloned into a vector, and propagated in E-coli. A restriction fragment can then be obtained from this construct. The restriction fragment should include the entire mouse Axs gene, as well as the 5′ and 3′ flanking sequences, respectively. This restriction fragment should be gel-purified, using conventional means so that the restriction fragment can be used for micro-injection into mouse zygotes. Founder animals, in which the cloned DNA fragment is randomly integrated into the genome, will be obtained at a frequency of 5%-30% of live born mice.

The presence of the transgene can then be ascertained by performing Southern blot analysis of genomic DNA, extracted from a small amount of mice tissue, such as the tip of a tail. DNA can be extracted using the following protocol: the tissue can be digested overnight at 55° C. in a lysis buffer, containing 200 mM NaCl, 100 mM Tris Ph 8.5, 5 mM of EDTA, 0.2% SDS, and 0.5 mg/mL of proteinase K.

The next day the DNA can be extracted once, with phenyl/chloroform (50:50), once with chloroform/isoamylalchohol (24:1) and precipitated with ethanol. This sample is then re-suspended in TE (10 mM Tris Ph 7.5, 1 mM EDTA). 8-10 μg of each DNA sample will then be digested, with a restriction endonuclease subject to gel-electrophoresis, and transferred to a charged nylon membrane. The resulting filter will then be hybridized with a radioactively labeled fragment of DNA, deriving from the mouse Axs gene locus, and able to recognize both a fragment from the endogenous gene locus and a fragment from a different size deriving from the transgene.

Once the presence of the DNA is confirmed, the founder animals are bred to normal non-transgenic mice, to generate sufficient numbers of transgenic and non-transgenic progeny, in which to determine the effects of Axs gene over-expression. For these studies, animals at various ages (for example, 1 day, 3 weeks, 6 weeks, and 4 months) are subject to a number of different assays designed to ascertain presence of the Axs protein. The transcriptional activity from the transgene may be determined by extracting RNA from various tissues, and using an RT-PCR assay, which takes advantage of single nucleotide polymorphisms between the mice strain from which the transgene is derived, and the strain of mice used for DNA micro-injection.

### Example 17

Homologous recombination and embryonic stem cells can be used to activate the endogenous mouse Axs gene, and subsequently generate animals carrying a loss of function mutation. A reporter gene, such as the E-coli, β-galactosidase gene, can be engineered into the targeting vector, so that its expression is controlled by the endogenous Axs gene's promoter and translational initiation signal. Thus, the spatial and temporal patterns of Axs gene expression can be determined in animals carrying the targeted allele.

A targeting vector must be constructed by the following process of Example 1 to form UAS constructs. The next step is to clone the targeting vector into a plasmid the same as stated in Example 1.

### Example 18

A kit can be prepared, which includes a container, which holds an isolated nucleic acid molecule, such as SEQ. ID NOs. 1-3. The isolated nucleic acid molecules can be used to form cDNA probes, which are included in the kit. The probes will also include labels, which will be inserted according to known procedures. The DNA sample can then be obtained from a non-human animal. This sample can be tested to determine if it is homologous to SEQ. ID NOs. 1-3 by using the kit. The sample DNA obtained from a non-human animal will be contacted with SEQ. ID NOs. 1-3 probes in the container of the kit. If the sample DNA attaches to the probe, it will indicate the presence of a mutant gene analog in the non-human mammal. This would indicate a high likelihood that spindle formation will not occur during female meiosis I, and disjunction will result. Non-hybridization indicates wild-type Axs.

### Example 19

A kit can be prepared, which includes a container, which holds an isolated wild-type nucleic acid molecule, such as SEQ. ID NO. 7. This isolated nucleic acid molecule can be used to form a cDNA probe, which is included in the kit. The probe will also include a label, which will be inserted according to known procedures. The DNA sample can then be obtained from a non-human animal. This sample can then be tested to determine if it is homologous to SEQ. ID NO. 7 by using the kit. The sample DNA obtained from a non-human animal will be contacted with the SEQ. ID NO. 7 probe in the container of the kit. If the sample DNA attaches to this probe, it will indicate the presence of a wild-type gene analog in the non-human animal. This would indicate a high likelihood that spindle formation will occur during female meiosis I, and disjunction will result.

### Example 20

A method can be practiced, whereby a mutant Axs protein can be used as part of a method, which serves to increase the likelihood of normal progeny from reproductively compromised individuals, including those individuals which exhibit a variety of chromosomally based diseases affecting meiosis. An oocyte from such individual can be withdrawn from the meiotic cell cycle and undergo atresia or apoptosis. In particular, individuals of a trisomic genotypic class exhibit segregation defects at a rate at least equal to, but likely greater than that observed in individuals with a normal genetic complement. One of the daughter products from these defective mitoses is what is considered to be a “normal” or wild-type genetic complement. Through these mitotic errors an individual trisomic for a particular chromosome would yield a normal disomic oocyte. The chromosomes are segregated by the normal chiasmate based system, and importantly, they are not sensitive to Axs^{D} or agents which phenocopy the effects of Axs^{D}. Thus, application of such agents serve as a strong selective agent leading to the preeminence of gametes with a normal chromosomal complement in individuals of this genotypic class.

The method is initiated by identifying female individuals that are predisposed to a trisomic genotype. After identifying a female, the Axs^{D} protein is introduced into the ova of the identified female, prior to ovulation. The protein will select against aneuploidy-generating ova. Application of Axs^{D} to an oocyte derived from a human of this genotypic class, results in the inhibition of proper meiotic spindle assembly in the oocyte.

### Example 21

A trisomic mutation can be identified through a combination of PCR amplification and direct sequencing of the mutant Axs or related genes. An individual predisposed for a mutation is identified. A germ cell is removed from the host individual. A target nucleotide sequence is isolated. PCR is used to amplify the sequence. The PCR product is then sequenced using known methods commercially available. The resultant information can be analyzed to determine the presence of a mutant. The sequence information is compared to a sequence of the wild-type gene. The female can be of any of a variety of origins, including of mammalian origin.

### Example 22

A kit for detecting the allelic state of an Axs or Axs related gene can be formed. Such a kit will consist of a container and a set of PCR primers spanning the Axs or Axs related gene in question. In addition, such a kit will contain a positive control consisting of a nucleic acid molecule representing a wild-type allele of the gene in question. For example, SEQ. ID NO. 7, the Axs cDNA or a genomic clone spanning the Axs gene can be used.

### Example 23

Example 20 can be further refined by removing an oocyte from an individual. The oocyte is treated in vitro with an Axs^{D} protein, such as that of SEQ. ID NO. 4. A sufficient amount of protein is added to cause nondisjunction in an oocyte having a trisomic chromosome. Gametes that do undergo meiosis will have a wild-type genotype. The gamete can be fertilized and implanted in the host from which it was removed.

What is claimed is:

1. An isolated nucleic acid molecule which inhibits female meiotic spindle assembly, selected from the group consisting of:
(a) an isolated nucleic acid molecule comprising SEQ. ID NOs. 1, 2, or 3, or complementary sequences thereof;
(b) degenerate variants of the sequences of step a; and,
(c) an isolated nucleic acid molecule that encodes an Axs^{D} protein according to (a) or (b).

2. An isolated nucleic acid molecule comprising a sequence at least 50% homologous to said nucleic acid molecules of claim 1(a).

3. An isolated nucleic acid molecule comprising a sequence at least 60% homologous to said nucleic acid molecules of claim 1(a).

4. An isolated nucleic acid molecule comprising a sequence at least 75% homologous to said nucleic acid molecules of claim 1(a).

5. An isolated nucleic acid molecule comprising a sequence at least 90% homologous to said nucleic acid molecules of claim 1(a).

6. An expression vector which prevents female meiotic spindle assembly comprising a promoter operably linked to a nucleic acid molecule according to any of claims 1 through 5.

7. A method of producing a protein that prevents female meiotic spindle assembly comprising culturing a cell which contains a vector according to claim 6, under conditions and for a time sufficient to produce said protein.

8. A viral vector capable of directing expression of a nucleic acid molecule according to claims 1 through 5.

9. A host cell carrying a vector according to any of claims 6 or 8.

10. An isolated oligonucleotide that binds to the nucleic acid molecule of claim 1.

11. An isolated protein which inhibits female meiotic spindle assembly, selected from the group consisting of:
(a) an isolated protein comprising SEQ. ID NOs. 4, 5, or 6;
(b) an Axs mutant allele protein encoded by the nucleic acid molecules of claim 1;
(c) an isolated protein that is 90% homologous to the protein of step a.

12. An antibody, which specifically binds to the proteins of claim 11.

13. A hybridoma that expresses the antibody of claim 12.

14. A probe for isolating a protein that promotes meiotic failure, wherein said probe is comprised of the antibody of claim 12.

15. The probe of claim 14, wherein said probe is at least 90% homologous to the protein of claim 7.

16. A cDNA probe comprising an isolated nucleic acid consisting of the nucleotide sequence of claim 1.

17. An antibody that binds specifically to Axs mutant allele protein.

18. An antibody that selectively binds to an epitope in the ligand-binding domain of the Axs mutant allele protein.

19. A method of inhibiting female meiotic spindle assembly comprising:
(a) expressing a mutant gene to form an Axs mutant allele protein; and,
(b) contacting said protein with an oocyte during Prophase I, in order to inhibit female meiotic spindle assembly.

20. The method of claim 19, wherein said oocyte is from Drosophila.

21. The method of claim 19, wherein said oocyte is from a non-human animal.

22. The method of claim 19, wherein expression is controlled by injection of an Axs mutant allele encoding nucleic acid molecule into an oocyte.

23. The method of claim 19, wherein expression is controlled by delivery of an Axs mutant allele nucleic acid molecule by micro-vessels.

24. The method of claim 19, wherein a small molecule binds to an endogenous Axs protein to create a defect parallel to that generated by the Axs mutant allele.

25. A method for predicting spindle formation during female meiosis I, comprising:
(a) isolating an Axs mutant allele gene;
(b) forming an Axs mutant gene probe; and,
(c) contacting said gene probe with DNA from a homolog, whereby hybridization of said probe indicates, in a female, presence of a mutant gene analog in the homolog of the Axs gene, thereby indicating a high likelihood that spindle formation will not occur during female meiosis I and nondisjunction will result.

26. The method of claim 25, wherein said female is of a Drosophila origin.

27. The method of claim 25, wherein said female is of a non-human mammalian origin.

28. A method for predicting spindle formation during female meiosis I, comprising:
(a) isolating an Axs gene;
(b) forming an Axs gene probe; and,
(c) contacting said gene probe with DNA from a homolog, whereby hybridization of said probe indicates, in a female, presence of a normal gene analog in the homolog of the Axs gene, thereby indicating a high probability that spindle formation will occur during female meiosis I and disjunction will result.

29. The method of claim 28, wherein said female is of a Drosophila origin.

30. The method of claim 28, wherein said female is of a non-human mammalian origin.

31. A method for predicting nondisjunction during female meiosis I, comprising:
(a) forming an Axs mutant allele antibody probe specific to mutant forms; and,
(b) contacting said antibody probe with an oocyte, whereby hybridization of said probe indicates the presence of a mutant gene, thereby indicating nondisjunction will occur.

32. A method of purifying Axs mutant allele protein from a biological sample containing Axs mutant allele protein, comprising:
(a) providing an affinity matrix comprising the antibody of claim 17, bound to a solid support;
(b) contacting the biological sample with the affinity matrix, to produce an affinity matrix-Axs mutant protein complex;
(c) separating the affinity matrix Axs mutant protein complex from the remainder of the biological sample; and,
(d) releasing the Axs mutant allele protein from the affinity matrix.

33. An isolated nucleic acid molecule which promotes female meiotic spindle assembly, selected from the group consisting of:
(a) an isolated nucleic acid molecule comprising SEQ. ID NO. 7, or complementary sequences thereof;
(b) degenerate variants of the sequences of step a; and,
(c) an isolated nucleic acid molecule that encodes an Axs protein according to (a) or (b).

34. An isolated nucleic acid molecule comprising a sequence at least 50% homologous to said nucleic acid molecules of claim 33(a).

35. An isolated nucleic acid molecule comprising a sequence at least 60% homologous to said nucleic acid molecules of claim 33(a).

36. An isolated nucleic acid molecule comprising a sequence at least 75% homologous to said nucleic acid molecules of claim 33(a).

37. An isolated nucleic acid molecule comprising a sequence at least 90% homologous to said nucleic acid molecules of claim 33(a).

38. An expression vector, which promotes female meiotic spindle assembly comprising a promoter operably linked to a nucleic acid molecule according to any of claims 33 through 37.

39. A method of producing a protein that promotes female meiotic spindle assembly comprising culturing a cell which contains a vector according to claim 38, under conditions and for a time sufficient to produce said protein.

40. A viral vector capable of directing expression of a nucleic acid molecule according to claims 33 through 37.

41. A host cell carrying a vector according to any of claims 38 or 40.

42. An isolated oligonucleotide that binds to the nucleic acid molecule of claim 33.

43. An isolated protein which promotes female meiotic spindle assembly, selected from the group consisting of:
(a) an isolated protein comprising SEQ. ID NO. 8;
(b) an Axs allele protein encoded by the nucleic acid molecules of claim 33;
(c) an isolated protein that is 90% homologous to the protein of step a.

44. An antibody, which specifically binds to the proteins of claim 43.

45. A hybridoma that expresses the antibody of claim 44.

46. A probe for isolating a protein that promotes meiotic disjunction, wherein said probe is comprised of the antibody of claim 44.

47. The probe of claim 46, wherein said probe is at least 90% homologous to the protein of claim 44.

48. A cDNA probe comprising an isolated nucleic acid consisting of the nucleotide sequence of claim 34.

49. An antibody that binds specifically to Axs allele protein.

50. An antibody that selectively binds to an epitope in the ligand-binding domain of the Axs allele protein.

51. A hybridization kit for detecting an Axs mutant allele gene, wherein said kit comprises:
(a) a container; and,
(b) a nucleic acid molecule comprising the nucleotide molecules of claim 1.

52. A hybridization kit for detecting an Axs mutant protein, wherein said kit comprises:
(a) a container; and,
(b) an antibody of claim 17.

53. A meiotic sheath protein structure comprising:
(a) a meiotic sheath protein; and
(b) a protein of claim 43 attached to said sheath protein, whereby disjunction is promoted.

54. A method for destroying defective oocytes, comprising:
(a) forming an Axs mutant allele antibody probe specific to mutant forms;
(b) contacting said antibody probe with an oocyte, whereby attachment of said probe indicates the presence of a mutant gene, thereby indicating nondisjunction will occur; and,
(c) performing a method, whereby the oocyte is destroyed.

55. A method for destroying non-wild type oocytes, comprising:
(a) using PCR to identify an Axs mutant; and,
(b) contacting a mutant oocyte with Axs mutant protein, prior to prophase I.

56. A method for rescuing defective oocytes predisposed to nondisjunction comprising:
(a) forming an Axs mutant allele antibody probe specific to mutant forms;
(b) contacting said antibody probe with an oocyte, whereby attachment of said probe indicates the presence of a mutant gene, thereby indicating nondisjunction will likely occur; and,
(c) contacting an Axs protein with the oocyte during meiosis, whereby the oocyte is rescued.

57. A hybridization kit for detecting an Axs gene, wherein said kit comprises:
(a) a container; and,
(b) a nucleic acid molecule comprising the nucleotide molecules of claim 33.

58. A hybridization kit for detecting an Axs protein, wherein said kit comprises:
(a) a container; and,
(b) an antibody of claim 43.

59. A method for predicting nondisjunction during female meiosis I, comprising:
(a) forming an Axs antibody probe specific to non-mutant forms; and,
(b) contacting said antibody probe with an oocyte, whereby attachment of said probe indicates the presence of a normal gene, thereby indicating disjunction will occur.

60. A method for causing nondisjunction comprising:
(a) expressing a mutant gene to form an Axs mutant allele protein; and,
(b) contacting said protein with an oocyte during prophase I, in order to inhibit female meiotic spindle assembly.

61. A method for causing nondisjunction comprising:
(a) expressing a mutant protein to form an Axs mutant allele protein; and,
(b) contacting said protein with an oocyte during prophase I, in order to inhibit female meiotic spindle assembly.

62. A method for selecting against trisomic individuals comprising:
(a) identifying oocytes which are potentially trisomic; and,
(b) contacting the oocyte with an amount of Axs^{D} protein, whereby the trisomic oocyte will undergo aneuploidy, and a mitotic error oocyte will have a wild-type genotype.

63. A kit for detecting an Axs gene, wherein said kit comprises:
(a) a container; and,
(b) a pool of nucleic acid molecule comprising the nucleotide molecules of claim 1.

64. A method for predicting nondisjunction during female meiosis I, comprising:
(a) using PCR to analyze a nucleic acid molecule and produce a PCR product;
(b) sequencing the PCR product; and
(c) comparing the sequence with a wild-type sequence.

65. A model system comprising:
(a) a Drosophila oocyte; and,
(b) a mutant Axs protein.

66. A kit for detecting Axs gene and Axs mutant genes comprising:
(a) PCR primers spanning an Axs gene or related Axs gene;
(b) a positive control; and,
(c) sequencing products.

67. A method for selecting against oocytes which have a trisomic chromosome, comprising:
(a) identifying a fly which has a trisomic genotype;
(b) harvesting the oocyte;
(c) treating the oocyte in vitro; and,
(d) selecting for a wild-type oocyte.


SEQUENCE LISTING (8 sequences)

SEQ ID NO: 1 - DNA, 2572 bp, Drosophila
ggaaagaagaggagattctagttaatttaaacaaattaaaaccaaattaattgtgacatatatgatttatttttgtagttgttgctgttgttgtgacagagaggcgaatcgtttcgataacatggcagcgatgacgtcacgcgccacgcagctgggacagcaacaaaaacaattggatttgaaccggcaagaactgaatattttggcattattattaaaaattcagtattttggcattggcgcgggccaagatacactcatacacgcaattagcacacacacgcacaccgcaagtgcgagcgagatagcaagcacttactcatgcgagcggagaagaaaagcaaaaacaaaataaacgaaactgagagaattctgcaatatcatattcggatgtggatgtgattgttatattttgttattagttcagcgacgccactcgtcgtcacccgtaaacgatgtccgaagatgccaagagtccaggaccacgcacacggaacatcatcgagaatcagctgttccgccggcagcgtagcctaaaattggaggcactgcagcgccagcggaccttggattccaacgatggggtggagggattgggcgcagatacggaacccttcgacaagacgcacatcgtcatcatctttacggagaaggccaagctaagacactgtcaggatgtggagaagatcattcaggagtttggcatccagaccacgctggagatcgttgggaaaaccgaaaagtatctctacctatcggccagcgtggatactctgctccgtttggccgatgccgccgagctggagaagatgaccaccacgcacagcatgcaaaagttcaatcacggctgcatctcggactttctactgcccgggatgggcaaagagcagatcctgcgctactgcgagatacctgttctcatcaaggacgtaatccaggacggcattaagtcctacgtgcaaaagggctacatagaggatatgtttcccctccacgatatcctgtatctcgaacgcttcaactggaacctgaaacgcaccaagctgcccatcgaggacatccggaactactttggttccagtataggtctttatttcggcttcatcgagttctacacgaaggcactgatctttccctccctttttggtatactccaatatgtattcgatctgaacatctcgctggtctgcagtttctacgttgtttggaccacgattttcctggagttgtggaagcgtaagtgtgccggctactcgtatcgatggggcaccatcgagatgagcagcctggacaagccgcgatccgcatatacgggccaattgaaaccggaccccatcaccggcaagatgacactccactatccgatgcggtacacatacctgcagatgtactgcatctcgtatccggtggttctgggctgtgtggttgccgccggctggtttgccctctaccagtttcagatcgaagccgaggtgctggcggatttcggaccagactcctggctgctgtacgtgccggttattgtgcagtcggtgctgattgcgatcttttcgtgggcatacgaaaagctggccacattcctcaccaacctggagaaccatcgaactcgatcgcagtacgatcgtcatcgggtcaataagctgatgctcttcaagatcgtgaataacttcttctcgcagttctatattgccttcgtgctgcacgatctgcgccagctgaagtaccagttgatgatgcaactgctggtcttccagctgctgtgcatcgcccaggagattggtataccgctgctggcagtgctgcgccagaagtacgccgagttccgtcatcgcgaggtggccgaggagaagctgcgatccatcagtgatctgccgcgctacgagcaatcgttctacgaatccggactagatgaatatcattccacgtacgaggactacctgcaggtatgcatccagtttggattcgtggtcctattcgccgccgttgccccatttgccgccattggagctctgctgaacaacgtctttgcggtgcacattgatatgtggaagctgtgcaacatctttaagcgaccatttgcaaggcgcgccaagaacatcggcgcctggcagctggctttcgagctgctctcagtgatgtcgttgcttagcaactgcggtctgctcttccttcagccgaatgtcaaggacttcttctctcactggctgccatcggtgccggatctttcgttcgtgatcttcgaacacttgctgctgggcctgaagtttctcatccacaaggttatccacgaaaggccgcgctgggtgcgcatcggactgctaaaggcggacttcgagaccagccaggctctcaagcaactcaaaaaattcaaggcggaggccaacaagatggcctgatgggccacaagatcgccggatctcccactccactccttttggtgctaatgaaaccagtccattttaaatgttattatttataaacatacgactaagcgcgtttaccgcgaatgttcgagaccaacggaagtaaggtgccttaaacctaaaacttcatataaatatgtccacagagt

SEQ ID NO: 2 - DNA, 2572 bp, Drosophila
ggaaagaagaggagattctagttaatttaaacaaattaaaaccaaattaattgtgacatatatgatttatttttgtagttgttgctgttgttgtgacagagaggcgaatcgtttcgataacatggcagcgatgacgtcacgcgccacgcagctgggacagcaacaaaaacaattggatttgaaccggcaagaactgaatattttggcattattattaaaaattcagtattttggcattggcgcgggccaagatacactcatacacgcaattagcacacacacgcacaccgcaagtgcgagcgagatagcaagcacttactcatgcgagcggagaagaaaagcaaaaacaaaataaacgaaactgagagaattctgcaatatcatattcggatgtggatgtgattgttatattttgttattagttcagcgacgccactcgtcgtcacccgtaaacgatgtccgaagatgccaagagtccaggaccacgcacacggaacatcatcgagaatcagctgttccgccggcagcgtagcctaaaattggaggcactgcagcgccagcggaccttggattccaacgatggggtggagggattgggcgcagatacggaacccttcgacaagacgcacatcgtcatcatctttacggagaaggccaagctaagacactgtcaggatgtggagaagatcattcaggagtttggcatccagaccacgctggagatcgttgggaaaaccgaaaagtatctctacctatcggccagcgtggatactctgctccgtttggccgatgccgccgagctggagaagatgaccaccacgcacagcatgcaaaagttcaatcacggctgcatctcggactttctactgcccgggatgggcaaagagcagatcctgcgctactgcgagatacctgttctcatcaaggacgtaatccaggacggcattaagtcctacgtgcaaaagggctacatagaggatatgtttcccctccacgatatcctgtatctcgaacgcttcaactggaacctgaaacgcaccaagctgcccatcgaggacatccggaactactttggttccagtataggtctttatttcggcttcatcgagttctacacgaaggcactgatctttccctccctttttggtatactccaatatgtattcgatctgaacatctcgctggtctgcagtttctacgttgtttggaccacgattttcctggagttgtggaagcgtaagtgtgccggctactcgtatcgatagggcaccatcgagatgagcagcctggacaagccgcgatccgcatatacgggccaattgaaaccggaccccatcaccggcaagatgacactccactatccgatgcggtacacatacctgcagatgtactgcatctcgtatccggtggttctgggctgtgtggttgccgccggctggtttgccctctaccagtttcagatcgaagccgaggtgctggcggatttcggaccagactcctggctgctgtacgtgccggttattgtgcagtcggtgctgattgcgatcttttcgtgggcatacgaaaagctggccacattcctcaccaacctggagaaccatcgaactcgatcgcagtacgatcgtcatcgggtcaataagctgatgctcttcaagatcgtgaataacttcttctcgcagttctatattgccttcgtgctgcacgatctgcgccagctgaagtaccagttgatgatgcaactgctggtcttccagctgctgtgcatcgcccaggagattggtataccgctgctggcagtgctgcgccagaagtacgccgagttccgtcatcgcgaggtggccgaggagaagctgcgatccatcagtgatctgccgcgctacgagcaatcgttctacgaatccggactagatgaatatcattccacgtacgaggactacctgcaggtatgcatccagtttggattcgtggtcctattcgccgccgttgccccatttgccgccattggagctctgctgaacaacgtctttgcggtgcacattgatatgtggaagctgtgcaacatctttaagcgaccatttgcaaggcgcgccaagaacatcggcgcctggcagctggctttcgagctgctctcagtgatgtcgttgcttagcaactgcggtctgctcttccttcagccgaatgtcaaggacttcttctctcactggctgccatcggtgccggatctttcgttcgtgatcttcgaacacttgctgctgggcctgaagtttctcatccacaaggttatccacgaaaggccgcgctgggtgcgcatcggactgctaaaggcggacttcgagaccagccaggctctcaagcaactcaaaaaattcaaggcggaggccaacaagatggcctgatgggccacaagatcgccggatctcccactccactccttttggtgctaatgaaaccagtccattttaaatgttattatttataaacatacgactaagcgcgtttaccgcgaatgttcgagaccaacggaagtaaggtgccttaaacctaaaacttcatataaatatgtccacagagt

SEQ ID NO: 3 - DNA, 2572 bp, Drosophila
ggaaagaagaggagattctagttaatttaaacaaattaaaaccaaattaattgtgacatatatgatttatttttgtagttgttgctgttgttgtgacagagaggcgaatcgtttcgataacatggcagcgatgacgtcacgcgccacgcagctgggacagcaacaaaaacaattggatttgaaccggcaagaactgaatattttggcattattattaaaaattcagtattttggcattggcgcgggccaagatacactcatacacgcaattagcacacacacgcacaccgcaagtgcgagcgagatagcaagcacttactcatgcgagcggagaagaaaagcaaaaacaaaataaacgaaactgagagaattctgcaatatcatattcggatgtggatgtgattgttatattttgttattagttcagcgacgccactcgtcgtcacccgtaaacgatgtccgaagatgccaagagtccaggaccacgcacacggaacatcatcgagaatcagctgttccgccggcagcgtagcctaaaattggaggcactgcagcgccagcggaccttggattccaacgatggggtggagggattgggcgcagatacggaacccttcgacaagacgcacatcgtcatcatctttacggagaaggccaagctaagacactgtcaggatgtggagaagatcattcaggagtttggcatccagaccacgctggagatcgttgggaaaaccgaaaagtatctctacctatcggccagcgtggatactctgctccgtttggccgatgccgccgagctggagaagatgaccaccacgcacagcatgcaaaagttcaatcacggctgcatctcggactttctactgcccgggatgggcaaagagcagatcctgcgctactgcgagatacctgttctcatcaaggacgtaatccaggacggcattaagtcctacgtgcaaaagggctacatagaggatatgtttcccctccacgatatcctgtatctcgaacgcttcaactggaacctgaaacgcaccaagctgcccatcgaggacatccggaactactttggttccagtataggtctttatttcggcttcatcgagttctacacgaagacactgatctttccctccctttttggtatactccaatatgtattcgatctgaacatctcgctggtctgcagtttctacgttgtttggaccacgattttcctggagttgtggaagcgtaagtgtgccggctactcgtatcgatggggcaccatcgagatgagcagcctggacaagccgcgatccgcatatacgggccaattgaaaccggaccccatcaccggcaagatgacactccactatccgatgcggtacacatacctgcagatgtactgcatctcgtatccggtggttctgggctgtgtggttgccgccggctggtttgccctctaccagtttcagatcgaagccgaggtgctggcggatttcggaccagactcctggctgctgtacgtgccggttattgtgcagtcggtgctgattgcgatcttttcgtgggcatacgaaaagctggccacattcctcaccaacctggagaaccatcgaactcgatcgcagtacgatcgtcatcgggtcaataagctgatgctcttcaagatcgtgaataacttcttctcgcagttctatattgccttcgtgctgcacgatctgcgccagctgaagtaccagttgatgatgcaactgctggtcttccagctgctgtgcatcgcccaggagattggtataccgctgctggcagtgctgcgccagaagtacgccgagttccgtcatcgcgaggtggccgaggagaagctgcgatccatcagtgatctgccgcgctacgagcaatcgttctacgaatccggactagatgaatatcattccacgtacgaggactacctgcaggtatgcatccagtttggattcgtggtcctattcgccgccgttgccccatttgccgccattggagctctgctgaacaacgtctttgcggtgcacattgatatgtggaagctgtgcaacatctttaagcgaccatttgcaaggcgcgccaagaacatcggcgcctggcagctggctttcgagctgctctcagtgatgtcgttgcttagcaactgcggtctgctcttccttcagccgaatgtcaaggacttcttctctcactggctgccatcggtgccggatctttcgttcgtgatcttcgaacacttgctgctgggcctgaagtttctcatccacaaggttatccacgaaaggccgcgctgggtgcgcatcggactgctaaaggcggacttcgagaccagccaggctctcaagcaactcaaaaaattcaaggcggaggccaacaagatggcctgatgggccacaagatcgccggatctcccactccactccttttggtgctaatgaaaccagtccattttaaatgttattatttataaacatacgactaagcgcgtttaccgcgaatgttcgagaccaacggaagtaaggtgccttaaacctaaaacttcatataaatatgtccacagagt

SEQ ID NO: 4 - protein, 646 aa, Drosophila
MSEDAKSPGPRTRNIIENQLFRRQRSLKLEALQRQRTLDSNDGVEGLGADTEPFDKTHIVIIFTEKAKLRHCQDVEKIIQEFGIQTTLEIVGKTEKYLYLSASVDTLLRLADAAELEKMTTTHSMQKFNHGCISDFLLPGMGKEQILRYCEIPVLIKDVIQDGIKSYVQKGYIEDMFPLHDILYLERFNWNLKRTKLPIEDIRNYFGSSIGLYFGFIEFYTKALIFPSLFGILQYVFDLNISLVCSFYVVWTTIFLELWKRKCAGYSYRWGTIEMSSLDKPRSAYTGQLKPDPITGKMTLHYPMRYTYLQMYCISYPVVLGCVVAAGWFALYQFQIEAEVLADFGPDSWLLYVPVIVQSVLIAIFSWAYEKLATFLTNLENHRTRSQYDRHRVNKLMLFKIVNNFFSQFYIAFVLHDLRQLKYQLMMQLLVFQLLCIAQEIGIPLLAVLRQKYAEFRHREVAEEKLRSISDLPRYEQSFYESGLDEYHSTYEDYLQVCIQFGFVVLFAAVAPFAAIGALLNNVFAVHIDMWKLCNIFKRPFARRAKNIGAWQLAFELLSVMSLLSNCGLLFLQPNVKDFFSHWLPSVPDLSFVIFEHLLLGLKFLIHKVIHERPRWVRIGLLKADFETSQALKQLKKFKAEANKMA

SEQ ID NO: 5 - protein, 269 aa, Drosophila
MSEDAKSPGPRTRNIIENQLFRRQRSLKLEALQRQRTLDSNDGVEGLGADTEPFDKTHIVIIFTEKAKLRHCQDVEKIIQEFGIQTTLEIVGKTEKYLYLSASVDTLLRLADAAELEKMTTTHSMQKFNHGCISDFLLPGMGKEQILRYCEIPVLIKDVIQDGIKSYVQKGYIEDMFPLHDILYLERFNWNLKRTKLPIEDIRNYFGSSIGLYFGFIEFYTKALIFPSLFGILQYVFDLNISLVCSFYVVWTTIFLELWKRKCAGYSYR

SEQ ID NO: 6 - protein, 646 aa, Drosophila
MSEDAKSPGPRTRNIIENQLFRRQRSLKLEALQRQRTLDSNDGVEGLGADTEPFDKTHIVIIFTEKAKLRHCQDVEKIIQEFGIQTTLEIVGKTEKYLYLSASVDTLLRLADAAELEKMTTTHSMQKFNHGCISDFLLPGMGKEQILRYCEIPVLIKDVIQDGIKSYVQKGYIEDMFPLHDILYLERFNWNLKRTKLPIEDIRNYFGSSIGLYFGFIEFYTKTLIFPSLFGILQYVFDLNISLVCSFYVVWTTIFLELWKRKCAGYSYRWGTIEMSSLDKPRSAYTGQLKPDPITGKMTLHYPMRYTYLQMYCISYPVVLGCVVAAGWFALYQFQIEAEVLADFGPDSWLLYVPVIVQSVLIAIFSWAYEKLATFLTNLENHRTRSQYDRHRVNKLMLFKIVNNFFSQFYIAFVLHDLRQLKYQLMMQLLVFQLLCIAQEIGIPLLAVLRQKYAEFRHREVAEEKLRSISDLPRYEQSFYESGLDEYHSTYEDYLQVCIQFGFVVLFAAVAPFAAIGALLNNVFAVHIDMWKLCNIFKRPFARRAKNIGAWQLAFELLSVMSLLSNCGLLFLQPNVKDFFSHWLPSVPDLSFVIFEHLLLGLKFLIHKVIHERPRWVRIGLLKADFETSQALKQLKKFKAEANKMA

SEQ ID NO: 7 - DNA, 2572 bp, Drosophila
ggaaagaagaggagattctagttaatttaaacaaattaaaaccaaattaattgtgacatatatgatttatttttgtagttgttgctgttgttgtgacagagaggcgaatcgtttcgataacatggcagcgatgacgtcacgcgccacgcagctgggacagcaacaaaaacaattggatttgaaccggcaagaactgaatattttggcattattattaaaaattcagtattttggcattggcgcgggccaagatacactcatacacgcaattagcacacacacgcacaccgcaagtgcgagcgagatagcaagcacttactcatgcgagcggagaagaaaagcaaaaacaaaataaacgaaactgagagaattctgcaatatcatattcggatgtggatgtgattgttatattttgttattagttcagcgacgccactcgtcgtcacccgtaaacgatgtccgaagatgccaagagtccaggaccacgcacacggaacatcatcgagaatcagctgttccgccggcagcgtagcctaaaattggaggcactgcagcgccagcggaccttggattccaacgatggggtggagggattgggcgcagatacggaacccttcgacaagacgcacatcgtcatcatctttacggagaaggccaagctaagacactgtcaggatgtggagaagatcattcaggagtttggcatccagaccacgctggagatcgttgggaaaaccgaaaagtatctctacctatcggccagcgtggatactctgctccgtttggccgatgccgccgagctggagaagatgaccaccacgcacagcatgcaaaagttcaatcacggctgcatctcggactttctactgcccgggatgggcaaagagcagatcctgcgctactgcgagatacctgttctcatcaaggacgtaatccaggacggcattaagtcctacgtgcaaaagggctacatagaggatatgtttcccctccacgatatcctgtatctcgaacgcttcaactggaacctgaaacgcaccaagctgcccatcgaggacatccggaactactttggttccagtataggtctttatttcggcttcatcgagttctacacgaaggcactgatctttccctccctttttggtatactccaatatgtattcgatctgaacatctcgctggtctgcagtttctacgttgtttggaccacgattttcctggagttgtggaagcgtaagtgtgccggctactcgtatcgatggggcaccatcgagatgagcagcctggacaagccgcgatccgcatatacgggccaattgaaaccggaccccatcaccggcaagatgacactccactatccgatgcggtacacatacctgcagatgtactgcatctcgtatccggtggttctgggctgtgtggttgccgccggctggtttgccctctaccagtttcagatcgaagccgaggtgctggcggatttcggaccagactcctggctgctgtacgtgccggttattgtgcagtcggtgctgattgcgatcttttcgtgggcatacgaaaagctggccacattcctcaccaacctggagaaccatcgaactcgatcgcagtacgatcgtcatcgggtcaataagctgatgctcttcgagatcgtgaataacttcttctcgcagttctatattgccttcgtgctgcacgatctgcgccagctgaagtaccagttgatgatgcaactgctggtcttccagctgctgtgcatcgcccaggagattggtataccgctgctggcagtgctgcgccagaagtacgccgagttccgtcatcgcgaggtggccgaggagaagctgcgatccatcagtgatctgccgcgctacgagcaatcgttctacgaatccggactagatgaatatcattccacgtacgaggactacctgcaggtatgcatccagtttggattcgtggtcctattcgccgccgttgccccatttgccgccattggagctctgctgaacaacgtctttgcggtgcacattgatatgtggaagctgtgcaacatctttaagcgaccatttgcaaggcgcgccaagaacatcggcgcctggcagctggctttcgagctgctctcagtgatgtcgttgcttagcaactgcggtctgctcttccttcagccgaatgtcaaggacttcttctctcactggctgccatcggtgccggatctttcgttcgtgatcttcgaacacttgctgctgggcctgaagtttctcatccacaaggttatccacgaaaggccgcgctgggtgcgcatcggactgctaaaggcggacttcgagaccagccaggctctcaagcaactcaaaaaattcaaggcggaggccaacaagatggcctgatgggccacaagatcgccggatctcccactccactccttttggtgctaatgaaaccagtccattttaaatgttattatttataaacatacgactaagcgcgtttaccgcgaatgttcgagaccaacggaagtaaggtgccttaaacctaaaacttcatataaatatgtccacagagt

SEQ ID NO: 8 - protein, 646 aa, Drosophila
MSEDAKSPGPRTRNIIENQLFRRQRSLKLEALQRQRTLDSNDGVEGLGADTEPFDKTHIVIIFTEKAKLRHCQDVEKIIQEFGIQTTLEIVGKTEKYLYLSASVDTLLRLADAAELEKMTTTHSMQKFNHGCISDFLLPGMGKEQILRYCEIPVLIKDVIQDGIKSYVQKGYIEDMFPLHDILYLERFNWNLKRTKLPIEDIRNYFGSSIGLYFGFIEFYTKALIFPSLFGILQYVFDLNISLVCSFYVVWTTIFLELWKRKCAGYSYRWGTIEMSSLDKPRSAYTGQLKPDPITGKMTLHYPMRYTYLQMYCISYPVVLGCVVAAGWFALYQFQIEAEVLADFGPDSWLLYVPVIVQSVLIAIFSWAYEKLATFLTNLENHRTRSQYDRHRVNKLMLFEIVNNFFSQFYIAFVLHDLRQLKYQLMMQLLVFQLLCIAQEIGIPLLAVLRQKYAEFRHREVAEEKLRSISDLPRYEQSFYESGLDEYHSTYEDYLQVCIQFGFVVLFAAVAPFAAIGALLNNVFAVHIDMWKLCNIFKRPFARRAKNIGAWQLAFELLSVMSLLSNCGLLFLQPNVKDFFSHWLPSVPDLSFVIFEHLLLGLKFLIHKVIHERPRWVRIGLLKADFETSQALKQLKKFKAEANKMA